(19) United States
(12) Patent Application Publication
(10) Pub. No.: US 2011/0177136 A1
(43) Pub. Date: Jul. 21, 2011

(54) METHOD AND PRODUCT

(76) Inventors: Anant Paradkar, Bradford (GB); Adrian Kelly, Bradford (GB); Phil Coates, Leeds (GB); Peter York, Ikley (GB)

(21) Appl. No.: 13/055,877

(22) PCT Filed: Jul. 27, 2009
(86) PCT No.: PCT/GB2009/050924

§ 371 Date: Mar. 28, 2011

(30) Foreign Application Priority Data
Jul. 26, 2008 (GB) 0813709.3

(51) Int. Cl. A61K 9/00; A61K 31/455; A61K 31/55; A61K 31/522; A61K 31/60; B29C 43/02

(52) U.S. Cl. 424/400; 514/355; 514/217; 514/263.34; 514/161; 264/109

(57) ABSTRACT

The present invention provides a method of producing a co-crystal, the method comprising the steps of providing a first substance and a second substance, wherein the first and second substances are compatible to form a co-crystal, mixing said first and second substances together, and exposing the mixture of said first and second substances to prolonged and sustained conditions of pressure and shear, sufficient to form a co-crystal of said first and second substance. The prolonged and sustained conditions of pressure and shear are preferably applied in an extrusion process. Associated compositions and uses thereof are also provided.

The present invention relates to a method useful for forming products which are useful in a pharmaceutical context, and products formed by such a method. The invention relates particularly, but not exclusively, to methods of forming co-crystal products using extrusion, and products obtained or obtainable via such methods.

Crystal engineering has been investigated recently as a means of tailoring the physicochemical properties of active agents. Its application to pharmaceuticals provides a new path for the systematic discovery of a wider range of multi-component structures containing an active pharmaceutical ingredient (API) by reconsidering the types of molecules and intermolecular interactions that can be used to form crystalline complexes with pharmaceuticals. Crystal engineering provides an interesting potential alternative approach available for the enhancement of drug solubility, dissolution and bioavailability.

A co-crystal can be considered to be a crystalline material made up of two or more components, usually in a stoichiometric ratio, each component being an atom, ionic compound or molecule held together by non-covalent forces. Co-crystals were previously referred to as binary compounds or molecular complexes. The physicochemical properties of the API and the co-crystal forming material properties can be modified, whilst maintaining the intrinsic activity of the drug molecule.

Pharmaceutical co-crystallisation is emerging as an attractive alternative to polymorphs, salts and solvates in the modification of an API during dosage form design (Blagden N., de Matas M., Gavan P. T., York P.; 2007 Crystal engineering of active pharmaceutical ingredients to improve solubility and dissolution rates. Advanced Drug Delivery Reviews. 59, 617-630). By co-crystallizing an active agent with a guest, one can create new solid state phases which may have improved properties over existing solid state phases of that active agent. For example, new drug formulations comprising co-crystals of active pharmaceutical ingredients (APIs) with pharmaceutically acceptable guests may have superior properties over existing drug formulations. In the pharmaceutical field, the active agent may be an API, and the other component of the co-crystal (the guest) must be a pharmaceutically acceptable compound (which could also be an API as appropriate). Active agents and guests may also include nutraceuticals, agricultural chemicals, pigments, dyes, explosives, polymer additives, lubricant additives, photographic chemicals, and structural and electronic materials. Physical properties of active agents, or their salts, may be modified by forming a co-crystal. Such properties include melting point, density, hygroscopicity, crystal morphology, loading volume, compressibility, and shelf life which may up-grade the performance of a drug formulation. Furthermore, other properties such as bioavailability, toxicity, taste, physical stability, chemical stability, production costs, and manufacturing method may be modified by using a co-crystal rather than the active agent alone, or as a salt. (Scott L. C., 2007. Co-crystallization methods. WO2007/038524).

Success rate of co-crystal formation is generally extremely low, because such a heteromeric system will only form if the non-covalent forces between two (or more) molecules are stronger than between the molecules in the corresponding homomeric crystals. Various crystal engineering techniques are currently under consideration for co-crystal design.

Solution crystallization is the most preferred method (Sudhakar P., Srivijaya R., Sreekanth B. R., Jayanthi P. K., Peddy Vishweshwar., Moses J. Babu., Vyas K., Javed Iqbal., 2007. Carboxylic acid-pyridine supramolecular heterocatemer in a co-crystal. Journal of Molecular Structure. In press; Renata Dreos., Lassaad Mechi., Giorgio Nardin., Lucio Randaccio., Patrizia Siega., 2005. Alternative co-crystallization of “almost” enantiomers and true enantiomers in some cis-b-organocobalt salen-type complexes with a-amino acids. Journal of Organometallic Chemistry. 690, 3815-382; Hickey B. Magali., Matthew L., Peterson A., Lisa A., et al., 2007. Performance comparison of a co-crystal of carbamazepine with marketed product. European Journal of Pharmaceutics and Biopharmaceutics. 67, 112-119; Scott L. C., Kenneth I. H., 2007. 4. Co-crystals of Piroxicam with Carboxylic Acids. Crystal Growth and Design. 1-14). This method is particularly of use to obtain single crystals for structure analysis—multiple samples of a chemical compound or element are solidified from solution under a variety of different solidification conditions. By varying one or more of temperature, solvent or anti-solvent content, seeds, concentration, agitation, purity, and other factors, one may create the conditions necessary to solidify solid forms from solution. Limitation of this method is the two components (active/guest) must have very similar solubilities; otherwise one component having least solubility will precipitate out. Also polymorph formation and apparent failure of this technique in a number of cases was observed.

In solid-state techniques such as grinding or milling, co-crystals are produced by grinding a mixture comprising at least two crystalline compounds (Xyrofin O. Y. 1996. Composition comprising co-crystals methods for its manufacture and its use. WO96/07331; Scott L. C., Kenneth I. H., 2007. 2. Co-crystals of Piroxicam with Carboxylic Acids. Crystal Growth and Design. 1-14).

Solution-based experiments produce a much wider variety of forms for each guest compared to grinding. As a screening technique grinding experiments are, however, a good complement to traditional solution-based experiments because they can identify forms not readily obtained from solution, but they are not a substitute for solution experiments. Emphasis has been given on the use of multiple experimental techniques when screening for co-crystals. Solid-state techniques such as grinding or milling are, however, labour intensive and are often difficult to perform in small vessels such as microliter well plates. But these latter empirical process developments are not well-understood and their success has lent an air of mystery to the preparative methodology of co-crystal formation (Chiarella R. A., Davey R. J., Peterson M. L., 2007. Making Co-Crystals—The Utility of Ternary Phase Diagrams. Crystal Growth and Design. Vol. 0, 1-4). But by selection of proper solvent during grinding on basis of polarity, formation of polymorphs can be controlled which is a significant drawback of co-crystallization techniques (Andrew V. Trask., W. D. Samuel Motherwellb and William Jones., 2004. Solvent-drop grinding: green polymorph control of cocrystallization. Chem. Commun. 890-891).

Screening techniques for solid forms by ultrasound crystallization and co-crystallization using ultrasound have been investigated (Scott L. C., 2005. Screening for solid forms by ultrasound crystallization and cocrystallization using ultrasound. WO/2005/089375). Sonication of a solution has been carried out to obtain co-crystals—the cavitational energy produced by the ultrasound appears to be useful for development of co-crystals. The importance of ultrasound in controlling supersaturation, nucleation and crystal growth have also been investigated (Hong Li., Hairong Li., Zhichao Guo., Yu Liu., 2006. The application of power ultrasound to reaction crystallization. Ultrasonics Sonochemistry 13, 359-363; 2. Ruecroft G., Hipkiss D., Tuan Ly., Maxted N., Peter W. Cains., 2005. Sonocrystallization: The Use of Ultrasound for Improved Industrial Crystallization. Organic Process Research and Development. 9, 923-932; Castro et al., 2007). Bucar et al. (Dejan-Kresimir Bucar., Leonard R. MacGillivray., 2007. Preparation and Reactivity of Nanocrystalline Cocrystals Formed via Sonocrystallization. J. Am. Chem. Soc. 129(1)) introduced sonocrystallization as a method to prepare organic co-crystals of nanosized dimensions and anticipate that this approach can be applied to other co-crystal systems to affect physical properties of bioactive materials (e.g., solubility). However, APIs or guests which are susceptible to oxidation can not be processed by this technique. The choice of solvent and anti-solvent pair is also critical.

U.S. Pat. No. 5,158,789 reports extrusion of the melts of two polyols, sorbitol and xylitol, which on solidificaton yield a product with the improved confectionary related properties.

Widespread industrial use of extruders has conventionally been in the plastics, rubber and food industries. In recent times the potential of extrusion has begun to be realised in pharmaceutical applications, largely because a number of functions can be performed in a single continuous operation. Therefore processes conventionally carried out by a number of separate batch operations can be combined, increasing manufacturing efficiency and potentially improving product consistency. However, extrusion based pharmaceutical process design has been developed from conventional plastics processing operations in conjunction with specialist feeding and downstream handling technology—it involves the dispersion of API into a polymeric matrix in a variety of forms. Most conventional polymer processing machinery can be adapted for use in a Good Manufacturing Practices (GMP) environment. Extrusion processing operations can be readily scaled from the laboratory to manufacturing scale and lend themselves well to in-process monitoring techniques, known within the pharmaceutical industry as Process Analytical Technology (PAT). Examples of pharmaceutical extrusion applications are briefly listed below:

Solid Dispersions:

Solid dispersions are defined as intimate mixtures of active drug substances (solutes) and diluents or carriers (solvent or continuous phase). In conventional technologies solid dispersions of drugs are typically produced by melt or solvent evaporation methods, where the materials produced are subsequently pulverised, sieved and mixed with excipients, before being encapsulated or compressed into tablets. Melt extrusion offers an improvement in manufacture of these systems, and has been used for particulate and molecular dispersions.

Controlled-Release Drug Delivery:

Controlled-released drug delivery systems offer numerous benefits over traditional dosage forms. The most common processes for the manufacture of controlled-release tablets include wet granulation and direct compression techniques, both of which are subject to content uniformity and segregation problems. Melt extrusion technology facilitates the design and development of controlled-release oral dosage forms without the use of water or solvents. Single or twin screw extruders with downstream micropelletisation or spheronisation capability are used to produce granules or extruded tablets. Hydrophillic and hydrophobic materials can be processed and only one component must melt or soften to facilitate material flow.

Transdermal Films:

Extruded sheet and film is used in the pharmaceutical industry, both for product packaging and for transdermal drug delivery systems. In the case of the latter application, an active ingredient is intimately mixed with a carrier and applied to a substrate. Conventional extruders are coupled with wide, thin dies in order to produce a continuous thin film wound directly on to water-cooled rollers, to thicknesses below 30 μm. Control of the take-up speed and roller temperature allows some degree of control of film crystallinity and molecular orientation. Multi layer films can be produced by co-extrusion, lamination or encapsulation.

Wet Granulation:

Granulation, in pharmaceutical terminology, refers to the process of particle formation via agglomeration of smaller particles. Granulation can be achieved through direct compaction, wet granulation or dry granulation. Wet granulation is traditionally carried out in batch mode by shearing a bed of powder blend with mechanical agitation, fluidization, or both. Twin screw extruders offer advantages over other methods of wet granulation due to the flexibility of screw design allowing control of the residence time and levels of distributive and dispersive mixing. This in turn permits control of the degree of agglomeration and homogenisation with relatively short process residence times, in the order of 1 minute. As a result, twin screw extrusion offers improved quality, space utilisation and reduced development time over conventional wet granulation methods.

Extrusion Spheronisation:

This is a multiple step process to make uniformly sized spherical particles which can be used in both immediate release and controlled release applications. These particles are used to fill gelatine capsules or compressed into tablets. The main advantage of extrusion spheronisation is the ability to incorporate high levels of active pharmaceutical ingredients within a relatively small particle. The extrusion spheronisation process combines the wet granulation process with the formation of spherical particles, drying, screening for size distribution and possibly coating. The spheronisation process transforms rod-shaped pellets product by extrusion into regular spherical particles by the action of a bowl with fixed walls and a rapidly rotating base with a grooved surface.

As touched on above, existing extrusion-based methods for use in pharmaceutical application are based upon the combination of an API with a polymeric carrier composition.

There is a need in the pharmaceutical field, and other fields, for improved methods of forming co-crystals.

According to a first aspect, the present invention provides a method of producing a co-crystal, the method comprising the steps of:
providing a first substance and a second substance, wherein the first and second substances are compatible to form a co-crystal;
mixing said first and second substances together; and
exposing the mixture of said first and second substances to prolonged and sustained conditions of pressure and shear sufficient to form a co-crystal of said first and second substance.

The term co-crystal refers to a composition which can be considered to be a crystalline material made up of two or more components; in particular the two or more components contribute to a crystal structure which is distinct from the crystal structures of the two components individually. Co-crystals can also be considered to be, and are sometimes referred to as, multi-component molecular crystals. A useful definition is: a co-crystal may be defined as a crystalline material that consists of two or more molecular (and electrically neutral) species held together by non-covalent forces, where both components are solids at room temperature (see Akeroy, Crystal engineering: strategies and architectures, Acta Cryst. B53 (1997) 569-586; and S. L. Morissette, O. Almarsson, M. L. Peterson, J. F. Remenar, M. J. Read, A. V. Lemmo, S. Ellis, M. J. Cima, C. R. Gardner, High Throughput crystallisation: polymorphs, salts, co-crystals and solvates of pharmaceutical solids, Adv. Drug Deliv. Rev. 56 (2004) 275-300.)

On a general level, it should be noted that co-crystals are distinct from solid dispersions. “Solid dispersion” is a generic term used to describe molecular and near molecular dispersion of compounds in an inert carrier. This includes simple eutectic mixtures and solid solutions. A simple eutectic mixture consists of two compounds which are completely miscible in the liquid state, but only to a very limited extent in the solid state. When a mixture of two molten materials is cooled, they both crystallize out simultaneously producing a physical mixture of very fine (but separate) crystals. Solid solutions are comparable to liquid solutions consisting of just one phase irrespective of the number of components. Classical solid solutions have a crystalline structure, in which the solute molecules substitute for solvent molecules in the crystal lattice. There is, however, no formation of a new crystal structure, as is the case with a co-crystal—the solute atoms merely substitute for molecules of the solvent without altering the structure or sit within the interstices between solvent molecules. In interstitial solid solutions, the volume of solute molecules is typically present at a level less than 20% of the solvent. This differentiates crystalline solid solutions from co-crystals, which demonstrate a defined systematic packing motif, accommodating guest and host molecules held together by non-covalent forces in stoichiometric ratios throughout the lattice to form a new crystal structure. In amorphous solid solutions, the solute molecules are dispersed molecularly, but irregularly, within the amorphous solvent. Solid dispersions can also encompass ultrafine crystalline drug particles dispersed in an amorphous or semi-crystalline matrix (see Improving drug solubility for oral delivery using solid dispersion, C. Leuner and J. Dressman, Eur. J. Pharm and Biopharm 50 (2000) 47-60).

Additionally, in respect of existing polymer and API extrusion technology, it should be clear that co-crystals have a different structure from both solid solutions and solid dispersion, both of which are common solid forms in polymer-based extrusion, discussed briefly above. In the context of polymer-based extrusion the former is an amorphous mix of API and polymer, the latter is a dispersion of API within a semi-crystalline polymer matrix.

The presence of co-crystals can be identified by a number of analytical techniques known to the person skilled in the art. The most rigorous of these is perhaps X-ray crystallography, which involves a detailed examination of the structure of a crystal, and potentially its resolution to single atom scale. However, X-ray crystallography is a time-consuming and complex process. Another, more time efficient and simpler, method for investigating crystal structure which is well suited to identifying co-crystal formation is the use of powder X-ray diffractometric (PXRD) characterization. In general the presence of co-crystal structures can be identified by the appearance of one or more new PXRD peaks. Amorphous forms exhibit a diffused powder X-ray diffraction pattern whereas co-crystals exhibit an additional characteristic peak/peaks which are not shown in the PXRD pattern of the single components or their physical mixtures. Through a knowledge of the PXRD patterns of the first and second substances (and in some instances the various polymorphic forms of these substances) it is possible to identify the presence of co-crystal structures by the appearance of one or more new PXRD peaks as compared with the first and second substances. Accordingly, embodiments of the present invention may involve the step of identifying the presence of a co-crystal by comparing the PXRD patterns of the output product of the method as set out above with the PXRD patterns of the first and second substances alone or in a mixture, or with known PXRD patterns of the co-crystal of interest.

It is preferable that the method of the present invention is a continuous flow method. The ability to perform a synthetic method in a continuous process is a significant advantage compared with conventional batch methods. Advantages over a batch process include improved efficiency, simpler scale-up, consistent product characteristics, and reduced need for cleaning.

In a preferred embodiment the first substance is an active pharmaceutical ingredient (API). As discussed above there is a particular need for improved methods for producing co-crystals in the pharmaceutical field. Existing technologies in this field suffer from disadvantages including being labour intensive, slow, inconsistent and/or unreliable, not amenable to scale-up or a combination of these problems. However, it should be noted that the present invention has application beyond the field of pharmaceuticals, including, for example, agrochemicals, explosives, nutraceuticals, pigments, dyes, lubricant additives, photographic chemicals, structural materials and electronic materials amongst others.

It is preferred that the first and second substances are exposed to sustained conditions of pressure and shear for at least 1 minute, preferably 2 minutes or longer, particularly 2 to 40 minutes, especially from 2 to 30 minutes. It will be appreciated that the length of time required to form co-crystals will generally depend on the severity of the pressure and shear conditions to which the first and second substances are exposed, but it has been found that prolonged and sustained exposure typically results in improved co-crystal formation. However, in many instances there will be a balance between possible API degradation though excessive time spent under shear and pressure conditions, against the amount of time required for co-crystal formation. Such a balance is a matter for optimisation depending on the materials used and the conditions imposed on these materials; such optimisation would typically be routine for the person skilled in the art.

It is preferred that the method is suitable to obtain an output product which comprises at least 20% w/w co-crystal, 40% w/w co-crystal, more preferably at least 60% w/w co-crystal, especially 80% w/w co-crystal. In methods according to the present invention it has been found that output products of 90% w/w or higher co-crystal purity can be achieved, which represents a remarkably high percentage purity in the production of co-crystals.

Accordingly, in highly preferred embodiments of the present invention the method is suitable to form output products comprising 90% w/w or higher co-crystal, preferably 95% or higher co-crystal, especially 99% or higher co-crystal. In general, it seems that higher percentage yields may be obtained by optimising the passage time and/or the intensity of the pressure and shear conditions.

In a preferred embodiment the pressure and shear are applied in an extrusion method. It is surprising that the process of extrusion can be used to obtain co-crystals which, as has been discussed earlier, are currently difficult entities to obtain. In addition, extrusion provides a method of producing high yields of co-crystals, and in large quantities. This provides very significant advantages over existing co-crystallisation technologies.

By extrusion it is meant the conveyance of the substances through an elongate lumen, while pressure and shear are applied; typically the pressure and shear are applied at least partially by means which conveys the substances through the lumen. The extrusion may also involve passing the substances through a die to shape or otherwise manipulate the product of the extrusion process, although this is generally not necessary for co-crystal formation.

It is generally preferred that the extrusion is a screw-based extrusion method. Although single screw extrusion may be suitable in some embodiments, it is generally preferred that the method is a screw-based extrusion method wherein two are more screws interact with the mixture of said first and second substances during the extrusion process. Such methods provide for a greater degree of mixing and otherwise manipulating the mixture to obtain the desired co-crystallisation.

In a preferred embodiment the screw-based extrusion method is a twin-screw extrusion method. Twin-screw methods provide a useful balance of minimising complexity of the extrusion apparatus, while providing the ability to manipulate the extrusion process as desired. It is, of course, possible that an extrusion process in which three or more screws interact may be used—and such systems are well known for the extrusion of polymers.

It is generally preferred that, where a twin-screw extrusion method is used, it is a co-rotating method. However, in some embodiments it may be found that a counter-rotating method provides some benefits.

Counter-rotating screws are used when very high shear is required, as they produce high pressures and shear forces between the two counter rotating screws. Thus counter-rotating screws may be useful where a very high level of shear and pressure is preferred to form the co-crystals. However, counter-rotating screw systems can suffer from problems with air entrapment, low maximum screw speeds and output; these may be disadvantages in certain applications.

Co-rotating systems can achieve a good level of mixing and conveying of materials and can also be operated at high speeds and thus achieve high output rates. They are less prone to wear than counter-rotating systems.

It is preferred that, where more than one screw is present, that the screws are at least substantially intermeshing, preferably fully intermeshing. A pair of screws can be considered to be fully intermeshing when the flight tip of helical threaded regions of each screw substantially reaches the root of the other screw; there will typically be a small gap to provide mechanical clearance, but generally the gap will be kept to a minimum. In an effort to quantify this term, it could be suggested that a pair of screws are substantially intermeshing when the gap between the flight tip of one screw and the root of the other is 10% or less of the total depth of the root of the screws, more preferably 5% or less. Intermeshing systems have the advantage that they are self-wiping and prevent localised overheating of materials within the system.

Of course, it should be noted that, in certain embodiments of the present invention, it may be preferable to use a non-intermeshing system. Non-intermeshing systems may be used where it is desired that large amounts of volatiles are removed from the system, or where highly viscous materials may result in unacceptably high levels of torque being applied to the system.

Another potential type of extruder for use in the present method is a recirculating extruder. Recycling extruders are typically twin-screw systems in which a batch of material can be processed for a predetermined period until being discharged from the system. Such extruders, for example the Haake Minilab, may be useful in a variety of applications, though are not as widely used as more conventional non-recirculating extruders. They are not generally as well suited to scale-up as conventional extruders, being a batch system. However, their ability to process small amounts, i.e. as low as 5 g, does make them suitable for some pharmaceutical studies.

It is generally preferred that the method is performed solely with the first and second substances which are capable of forming a co-crystal, especially with an API and a co-crystal former or “guest” material (which could itself be an API). It should be noted that it is possible that more than 2 substances could co-operate to form a co-crystal, and thus additional co-crystal forming substances could be present. It is thus preferred that the method of the present invention is performed substantially in the absence of any non-crystal forming materials, for example, solvents or lubricants. It is a significant advantage of the method of the present invention that co-crystals can be formed in the absence of solvents or other additional substances. Removal of such additional substances following production can be difficult or impossible, and the presence of such substances, even at low levels, can be a significant safety concern, or at least a regulatory hurdle. Of course it will be understood that there may be circumstances where the presence of, e.g. a lubricant or solvent, might be desirable, and the method of the present invention is compatible with the inclusion of such additives, but it is a significant advantage that they can be omitted from the method.

For the avoidance of doubt, it should be mentioned that the materials involved forming the co-crystal should be capable of forming a crystalline structure. Thus polymers which form amorphous or “semi-crystalline” structures are not suitable for forming a co-crystal in the process of the present invention.

It may be desirable that the mixture of said first and second substances is exposed to additional heat. By “additional heat” it is meant that the mixture has heat applied to it, beyond the ambient temperature and beyond the heat produced by friction during the extrusion process.

In certain embodiments it is preferred that the process is carried out, for at least a portion of the duration of the process, at a temperature around the melting point of the co-crystal forming substance with the lowest melting point. In general it is preferred that the temperature is slightly below the melting point of the co-crystal forming substance with the lowest melting point, though it might be at or slightly above the melting point. In preferred embodiments the temperature may be within 20° C. of the melting point, preferably within 10° C. of the melting point. In the case where one of the components is a eutectic mixture, the relevant melting point would be that of the mixture. It has been found that where such a temperature is used, there is a benefit in terms of co-crystal formation.

Depending on the conditions of shear and pressure and dwell time required during the extrusion process to obtain co-crystals of the desired properties and yield, the configuration of the screw or screws can be altered. In general twin-screw or other multiple screw arrangements are more amenable to modification of configuration, but it is possible to a lesser extent with a single screw extruder. It is possible to alter the following aspects of the extrusion apparatus or process, amongst others: length of barrel, ratio of length:diameter of the barrel (LID ratio), composition of the screw elements (e.g. dispersive or distributive mixing elements, forward or reverse feed elements, depth of screw root (i.e. thread depth), screw rotation speed, feeding method (starvation feed versus flood feeding), number of passes through the extruder. These aspects enable a high degree of control over the extrusion process and the resultant co-crystal product.

It has been found that during extrusion it is preferable that the L/D ratio is 15/1 or greater (i.e. length is 15× or greater than the diameter of the screw). Preferably the L/D ratio is 20/1 or greater, and in some embodiments a ratio of 30/1 or greater may be preferable. An LID ratio of 40/1 has been found to be well suited to formation of co-crystals. These ratios apply especially to twin-screw systems, but can also apply to other extrusion systems.

It is preferred that during extrusion the mixture is exposed to at least one period of distributive or dispersive mixing. It is generally preferable that the mixture is exposed to at least one period of dispersive mixing; dispersive mixing is more aggressive in terms of shear, pressure and heat production, and thus appears to often be useful in driving the formation of co-crystals. Generally it is most preferred that the mixture is exposed to at least one period of each of distributive and dispersive mixing.

The screw of an extruder, especially a twin-screw or other multiple screw extruder, can comprise a number of different elements which determine the conditions to which the substances are subjected during extrusion. It should be noted that these elements are not always “screws” in the strictest sense, in that they may not comprise a continuous helical thread, but the term screw is nonetheless used in relation to the assembly as a whole regardless of the composition. Generally a significant portion of the length of a screw will comprise helical threads, typically half or more of its length will comprise helical threads.

The elements which make up the screw are typically assembled onto a shaft to form the complete screw. The shaft typically has a cross-section which prevents rotation of the elements relative to the shaft, e.g. polygonal, and in many instances hexagonal. Each element is typically quite short relative to the total length of the screw. It is most convenient to talk of the length of the elements in terms of proportion of the diameter of the screw of the extruder.

Helical screw elements are used to convey the substances through the extruder, and they confer a relatively low level of mixing and application of pressure and shear. The level of pressure and shear applied by such helical elements can be varied, for example, by varying the degree of intermeshing of such helical elements in a multiple screw extruder, and varying the depth and/or pitch of such elements. Different helical screw types may be present, for example forward conveying elements, discharge elements or reverse screw elements.

Where more intense mixing and application of shear and pressure are required, this can be achieved by using mixing elements, especially mixing paddles. Mixing paddles typically comprise lobed elements, e.g. elliptical or similar shaped elements, which do not comprise a helical thread. The paddles provided a curved flat mixing surface. In a twin-screw extruder one or more corresponding pairs of lobed elements may be provided on each of the screws. The lobed element on one screw is arranged such that it is rotationally offset relative to the lobed element on the other screw, typically by 90° for bi-lobed (i.e. generally elliptical) paddles, such that when the elements rotate the mixing surfaces of the lobed elements are separated by a narrow gap, which may remain substantially constant during rotation due to the corresponding shapes of the pair of paddles, or might vary to some degree during rotation. Different degrees of offset may be used for tri-lobed, or other shapes of mixing elements as appropriate. The effect of such mixing paddles is that the mixture is smeared between the pair of paddles and is thus subjected to relatively intense mixing at high shear and pressure. In addition, the flat nature of the mixing surface means that forward conveyance is not strongly promoted and, as such, the mixture tends to dwell in such elements; forward conveyance of the mixture is primarily driven by pressure exerted by the upstream mixture being forced by upstream conveying elements, although, as discussed below, certain configurations of mixing elements can provide a degree of forward conveyance.

The degree of mixing and application of shear and pressure can be determined by the number and configuration of mixing elements. Distributive mixing is a term well known in the art of extrusion and can be defined as—“distributive mixing is the process of spreading a minor component throughout a matrix in order to achieve good spatial distribution”. Distributive mixing can be achieved by providing a sequence of pairs of mixing (e.g. lobed) elements, where each pair of mixing elements is rotationally offset relative to the preceding pair, i.e. at staggered angles. Generally subsequent mixing elements are offset in the same direction as the direction of the helical portion which provide forward conveyance. Typically the length of each mixing element (e.g. lobed element) will be up to 0.25× the diameter of the screw, preferably at least 0.125× the diameter of the screw; e.g. for a screw of diameter 16 mm, each element might have a length of 4 mm. Distributive mixing can be considered to be mixing predominantly by rearranging flow paths of the mixture of the substances; in essence the relative short length of each mixing element means that the mixture is churned between the mixing elements, and the level of highly constrained smearing is relatively low. The amount of rotational offset determines the amount of conveyance such a distributive mixing sequence provides, and to some extent the severity of the mixing. Where a pair is offset from the preceding pair by from around 10° to 45° (typically)30° in the same direction as the helix on the feed screw, a significant degree of forward conveyance is provided; an offset of from around 46° to 65° (typically)60° provides somewhat less conveyance; and an offset of from around 75° to 90° provided significantly less conveyance—an offset of 90° provides essentially no conveyance of the mixture.

Dispersive mixing is an intense form of mixing and provides a high level of shear and pressure to the mixture. Dispersive mixing is a term well known in the art of extrusion and can be defined as—“dispersive mixing involves the reduction in size of a cohesive minor component such as clusters of solid particles or droplets of a liquid”. Dispersive mixing can be achieved when the mixture is forced to pass through an elongate mixing region where it is compressed and smeared between mixing surfaces of mixing elements. Dispersive mixing can be provided by one or more mixing elements, e.g. bi-lobed elements, which provide an elongate region of mixing surface without any rotational offset; i.e. the elongate region of mixing surface may be provided by a pair of comparatively long mixing elements (one on each screw of a twin-screw system) with substantially no rotational offset, or there may be a plurality of sequential shorter mixing elements which have substantially no rotational offset between subsequent elements. For example, a region of 0.5× the diameter of the screw or greater in length comprising lobed mixing elements with no rotational offset will provide dispersive mixing. Conveniently a dispersive mixing zone can comprise two or more lobed elements which are not offset relative to one another, i.e. they provide a substantially continuous mixing surface. In essence the significant aspect of dispersive mixing is that at least a portion of mixture is constrained to pass through mixing elements between which the mixture is smeared and a high degree of pressure and shear is applied—this can be achieved using mixing elements as discussed above.

However, it should be noted that the above distributive and dispersive mixing systems are illustrative of preferred systems for use in the present invention. Other methods of achieving distributive or dispersive mixing could be envisaged by the person skilled in the art. A discussion of dispersive and distributive mixing is provided in Rheology Bulletin Vol. 66, No. 1 (January 1997)—Analysis of Mixing in Polymer Processing Equipment by Ica Manas-Zloczower.

It is preferred that the extrusion apparatus used in the present method comprises dispersive mixing regions (i.e. regions comprising mixing elements) for at least 1/40 of the total length of the screw, preferably at least 1/30, more preferably at least 1/20 of the total length of the screw. Preferably there is at least one region of dispersive mixing, the region being at least 0.5 diameters in length. More preferably there is at least one region of dispersive mixing, and the total length of all the regions of dispersive mixing is at least 1.5 diameters or more, preferably 2 diameters or more.

In embodiments of the present invention it is preferred to have both mixing and dispersive mixing regions, i.e. regions comprising mixing elements. In a preferred embodiment the configuration comprises at least one region of distributive mixing followed by at least one region of dispersive mixing. In a preferred embodiment of the invention, at least two regions of distributive mixing and at least two regions of dispersive mixing are provided. It is preferred that each of the regions of distributive mixing are at least 1 diameter in length, more preferably at least 1.5 diameters in length, and they may be 2 or more diameters in length. It is preferred that each of the regions of dispersive mixing are at least 0.5 diameters in length, they may be 1 or more diameters in length, and they may be 1.5 or more diameters in length. Generally it is preferred that there is a total of 5 or more diameters in length of mixing regions, more preferably 10 or more diameters of mixing regions.

It is generally preferred that half of the total screw length or less of the extrusion system comprises mixing elements. More typically ⅖ or less, or ¼ or less of the total screw length comprises mixing elements. Of course the actual proportion may vary depending on the total screw length, and situations where more than half the total length comprises mixing elements can be envisaged.

As mentioned above the first and second substances must be capable of forming a co-crystal. There are no definite rules yet available which predict the formation of co-crystals. However, in general it can be said that the first and second substances should possess complimentary groups with notable potential for hydrogen bond formation. Substances which typically fall into the desired category include carboxylic acids, amines, amides, sulphonamides, hydroxyl alcohols, ketones, amino acids, sugars, and heterocyclic bases, and accordingly such substances, or substances including appropriate active groups are preferred for use in the present method. In general it will be desired to form a co-crystal of a particular API, and it will be necessary to select a suitable guest or co-former for the co-crystal formation; i.e. there will be freedom to change the guest, but not usually the API. Identifying a suitable guest will typically involve a degree of trial and error, but will often be guided by a knowledge of the chemistry of the API. In general, however, APIs and guests which contain carboxylic acids, amides and heterocyclic bases are preferred for use in the present invention.

Examples of suitable APIs are set out in Table 1 below.

TABLE 1

| Primary Active Group | API Name |
|---|---|
| —COOH | Naproxen, ibuprofen, ketoprofen, tolmetin, |
| | fenoprofen, indomethacin, salicylic acid. |
| —C═O | Nabumetone, piroxicam, pioglitazone, glipizide, |
| | glimepiride, tolbutamide, warfarin, atorvastatin |
| | (statins), prazosin, captopril, nifedipine, |
| | felodipine, amlodepine, lidocaine. |
| —NH₂ | Lamotrignine, amphetamine, metformine, |
| | fluoxetine, primaquine, caffeine, theophylline. |
| —CONH₂ or | Carbamazepine, celecoxib, valdecoxib. |
| —CSO₂NH₂ | |
| —OH | Atenolol, propranolol. |

Examples of suitable guests are set out in Table 2 below.

TABLE 2

| Type | Example |
|---|---|
| Carboxylic acids, | Glutaric acid, citric acid, fumaric acid, malonic acid, |
| Dicarboxylic | oxalic acid, benzoic acid, malic acid, maleic acid, |
| acids | tartaric acid, succinic acid, adipic acid, salicylic acid, |
| | cinnamic acid, anthranilic acid, hippuric acid. |
| Amino acids | Tyrosine, lysine, arginine, isoleucine, tryptophan, |
| | histidine, cystine. |
| Sugars | Saccharin, fructose, mannitol, glucose, aspartame. |
| Amides | Nicotinamide. |

In embodiments of the present invention, it may be preferred that the first and second substances are provided in a stoichiometric ratio. The ratio may be 1:1 or it may be another integer ratio, such as 1:2, 2:1, etc.

In an embodiment of the present invention the first substance is a phenylalkanoic acid. Preferably the first substance is ibuprofen and the second substance is nicotinamide. Suitably the w/w ratio of ibuprofen to nicotinamide is approximately 41.2:26, i.e. a 1:1 molar ratio.

In another embodiment of the present invention the first substance is carbamazepine and the second substance is saccharin. Suitably the w/w ratio of carbamazepine to saccharin is approximately 47:37, i.e. a 1:1 molar ratio.

In another embodiment of the present invention the first substance is carbamazepine and the second substance is nicotinamide. Suitably the w/w ratio of carbamazepine to nicotinamide is approximately 118:61, i.e. a 1:1 molar ratio.

In another embodiment of the present invention the first substance is caffeine and the second substance is maleic acid. Suitably the w/w ratio of caffeine to maleic acid is approximately 194:116, i.e. a 1:1 molar ratio, or 97:29, i.e. a 2:1 molar ratio.

In another embodiment of the present invention the first substance is theophylline the second substance is maleic acid. Suitably the w/w ratio of theophylline to maleic acid is 45:29, i.e. a 1:1 molar ratio.

In another embodiment of the present invention the first substance is salicylic acid the second substance is nicotinamide. Suitably the w/w ratio of salicylic acid to nicotinamide is 69:61, i.e. a 1:1 molar ratio.

It is a notable aspect of the present invention that the product of the extrusion process is typically particles comprising agglomerates of co-crystals. These particles typically have a size of from 2 to 3000 μm in their largest dimension, and are very well suited to being compacted directly into tablet or other unit dose form. Furthermore, it has been demonstrated that the agglomerates dissolve readily in both water and in an in vitro model of the stomach, which is extremely useful in drug delivery.

Accordingly, in a further aspect the present invention provides a method of forming such particles comprising agglomerated co-crystals comprising the methods as set out above.

In another aspect the present invention provided particles of agglomerated co-crystals, preferably having a diameter of 2 to 3000 μm. Preferably the particles comprise at least 50% w/w co-crystal, more preferably at least 75% w/w co-crystal, and they may comprise 90% or more w/w co-crystal.

Optionally the method may include the step of introducing a modifier compound into the extrusion process. Suitable modifiers include density modifiers (lactose, microcrystalline cellulose, etc), binders (starch, celluloses, polyvinyl pyrollidone, etc), disintegrants (sodium starch glycollate, crosslinked polyvinyl pyrollidone), and wetting agents. Such modifiers may suitably be introduced to the extrusion process after co-crystallisation has substantially completed. As such, the modifiers are incorporated into the output product of the process, but do not interfere with the co-crystallisation process. For example, the modifier might be introduced downstream of all regions of dispersive mixing.

In a further embodiment the present invention provides a method of forming a unit dose form of an active agent comprising carrying out the method as set out above to form particles of agglomerated co-crystals, and compressing said particles, optionally in a suitable mould, to form the unit dose form. Suitably the unit dose is a tablet or similar. Optionally the method may involve providing a pharmaceutically acceptable excipient. Suitable excipients might include the compounds discussed above as modifiers.

In another aspect the present invention provides composition comprising a co-crystal, the co-crystal comprising a first and a second substance, wherein the first and second substance have been exposed to a process as set out above.

In one embodiment the present invention provides a composition comprising a co-crystal of a phenylalkanoic acid and nicotinamide, preferably ibuprofen and nicotinamide. The presence of the co-crystal can be identified by the presence of a characteristic PXRD peak of co-crystal at 3.2° 2-theta. Preferably the product comprises at least 50% co-crystal w/w, more preferably at least 75% co-crystal w/w, especially at least 90% co-crystal w/w.

In one embodiment the present invention provides a product comprising a co-crystal of carbamazepine and saccharin. The presence of the co-crystal can be identified by the presence of a characteristic PXRD peak of co-crystal at 7° 2-theta. Preferably the product comprises at least 50% co-crystal w/w, more preferably at least 75% co-crystal w/w.

In one embodiment the present invention provides a product comprising a co-crystal of carbamazepine and nicotinamide. The presence of the co-crystal can be identified by the presence of a characteristic PXRD peak of co-crystal at 20.4 2-theta. Preferably the product comprises at least 50% co-crystal w/w, more preferably at least 75% co-crystal w/w.

In one embodiment the present invention provides a product comprising a co-crystal of caffeine and maleic acid at a 1:1 molar ratio. The presence of the co-crystal can be identified by the presence of characteristic PXRD peaks of co-crystals at 9, 11.1, 13.2, 14.2, 15.5 and 13.2 2-theta. Preferably the product comprises at least 50% co-crystal w/w, more preferably at least 75% co-crystal w/w.

In one embodiment the present invention provides a product comprising a co-crystal of caffeine and maleic acid at a 2:1 molar ratio. The presence of the co-crystal can be identified by the presence of characteristic PXRD peaks of co-crystals at 8.8, 10.1, 13.5 and 16 2-theta. Preferably the product comprises at least 50% co-crystal w/w, more preferably at least 75% co-crystal w/w.

In one embodiment the present invention provides a product comprising a co-crystal of theophylline and maleic acid. The presence of the co-crystal can be identified by the presence of characteristic PXRD peaks of co-crystals at 9, 11.5, 13.6 and 16.8 2-theta. Preferably the product comprises at least 50% co-crystal w/w, more preferably at least 75% co-crystal w/w.

In one embodiment the present invention provides a product comprising a co-crystal of salicylic acid and nicotinamide. The presence of the co-crystal can be identified by the presence of characteristic PXRD peaks at 7.8, 8.4 and 9.1 2-theta. Preferably the product comprises at least 50% co-crystal w/w, more preferably at least 75% co-crystal w/w.

In a further aspect the present invention provides composition comprising a co-crystal obtained or obtainable by the process set out above.

In a further aspect the present invention provides a pharmaceutical preparation comprising a co-crystal as set out above, optionally in combination with a pharmaceutically acceptable excipient. The pharmaceutical preparation may be in any suitable form for administration, for example, granules, tablets, capsules or the like.

In a further aspect the present invention provides a composition comprising a co-crystal of ibuprofen and nicotinamide for the alleviation of pain or the treatment of an inflammatory condition. Examples of inflammatory conditions include trauma-induced inflammation and autoimmune conditions, for example, rheumatoid arthritis, lupus erythematosus, myasthenia gravis, pemphigus, Sjögren's syndrome, ankylosing spondylitis, inflammatory bowel disease, etc. Further details of a suitable co-crystal are set out above.

In a further aspect the present invention provides a composition comprising a co-crystal of carbamazepine and saccharin or carbamazepine and nicotinamide for the treatment of psychological disorders, such as epilepsy, bipolar disorder attention deficit disorder (ADD) or attention-deficit hyperactivity disorder (ADHD), schizophrenia, phantom limb syndrome and trigeminal neuralgia. Further details of a suitable co-crystal are set out above.

In a further aspect the present invention provides a composition comprising a co-crystal of caffeine and maleic acid for the treatment of disorders of the central nervous system of the respiratory system.

In a further aspect the present invention provides a composition comprising a co-crystal of theophylline and maleic acid for the treatment of chronic obstructive diseases of the airways (e.g. COPD), asthma (especially bronchial) or apnea (especially infant apnea).

In a further aspect the present invention provides a composition comprising a co-crystal of salicylic acid and nicotinamide for use in the alleviation of pain, the reduction of fever, or for the treatment of acne, psoriasis, callouses, corns, keratosis pilaris, and warts (e.g. via topical administration).

In a further aspect the present invention provides particles comprising agglomerated co-crystals obtainable or obtained according to the methods set out above. Such particles have desirable properties in terms of dissolution and compactability.

In a further aspect the present invention provides a composition comprising a co-crystal as set out above for use in a medical treatment. The present invention also provides the use of a composition comprising a co-crystal as set out above in the manufacture of a medicament for use in a medical treatment of one or more medical conditions mentioned above.

Embodiments of the present invention will now be described, by way of example only, with reference to the accompanying figures in which:

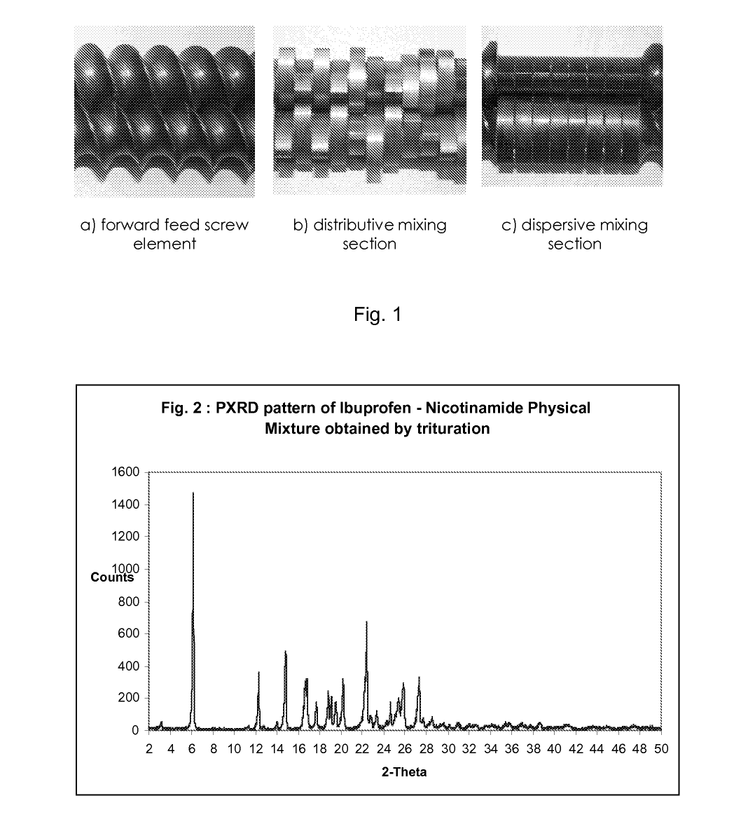

FIG. 1 shows examples of twin screw extruder screw elements;

FIG. 2 shows the PXRD pattern of physical mixture containing ibuprofen and nicotinamide;

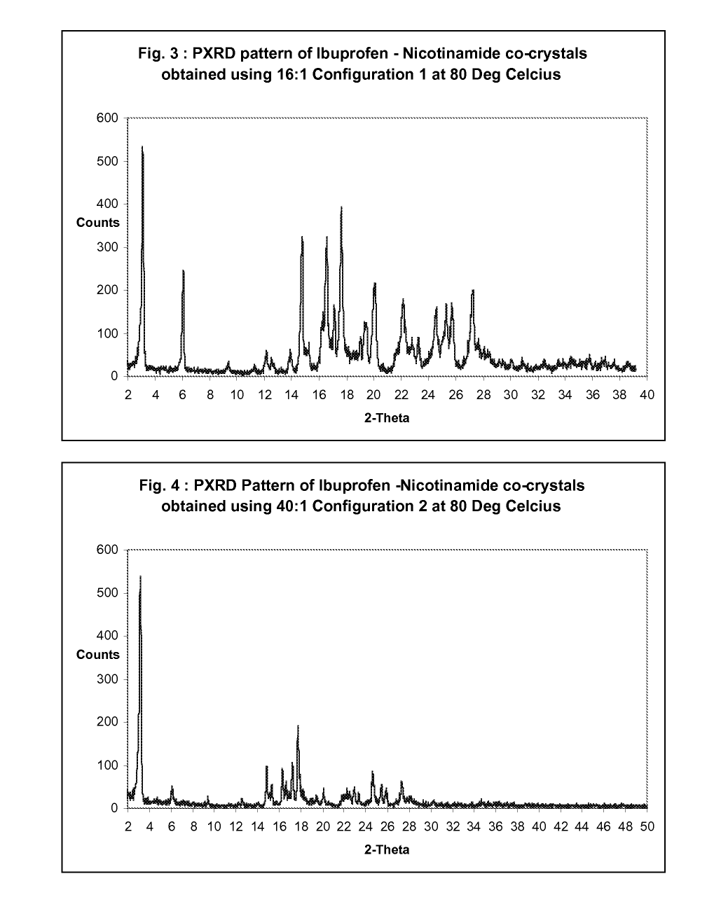

FIG. 3 shows the PXRD pattern of co-crystals of ibuprofen and nicotinamide obtained after extrusion using a 15:1 screw configuration with alternative feed and distributive mixing zones;

FIG. 4 shows the PXRD pattern of co-crystals of ibuprofen and nicotinamide obtained after extrusion using the 40:1 screw configuration with alternative feed and distributive mixing zones;

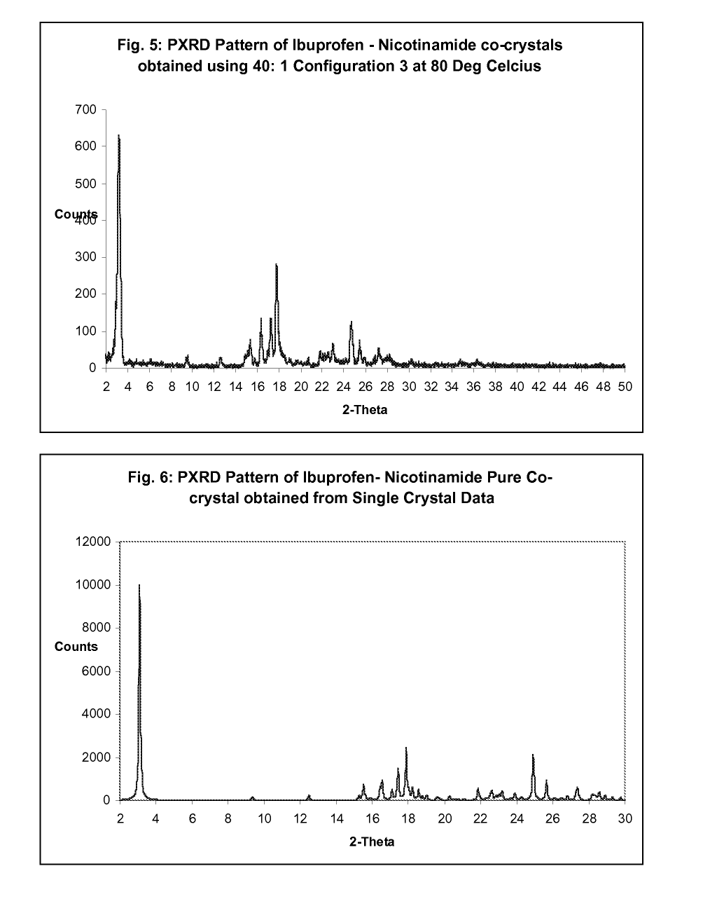

FIG. 5 shows the PXRD pattern of co-crystals of ibuprofen and nicotinamide obtained after extrusion using 40:1 screw configuration with feeding, distributive and dispersive mixing zones;

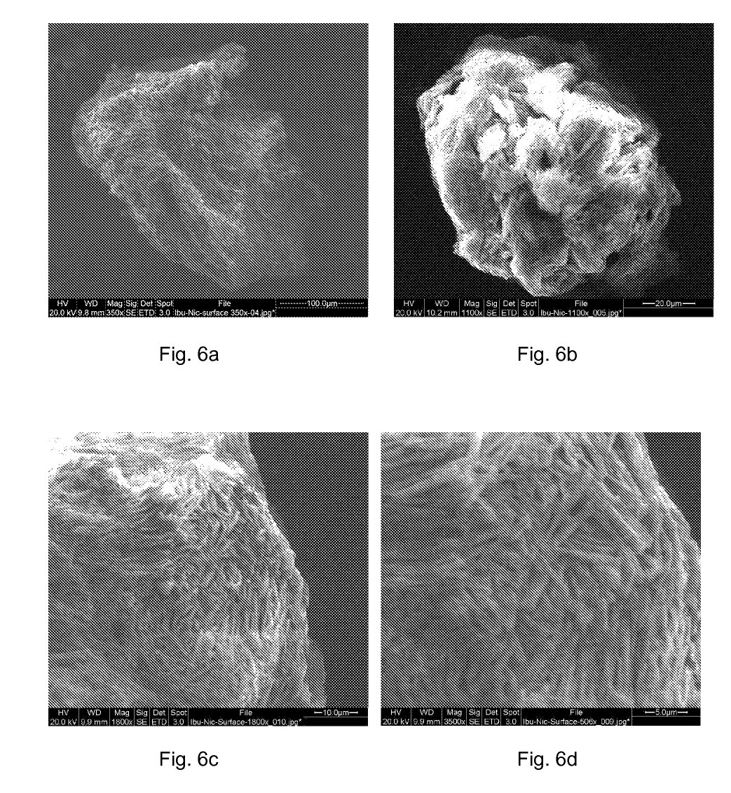

FIG. 6 shows the PXRD pattern obtained using the single co-crystal of ibuprofen and nicotinamide;

FIGS. 6a to 6d show SEM images of agglomerated ibuprofen and nicotinamide co-crystals at 350×, 1100×, 1800× and 3500× magnification respectively;

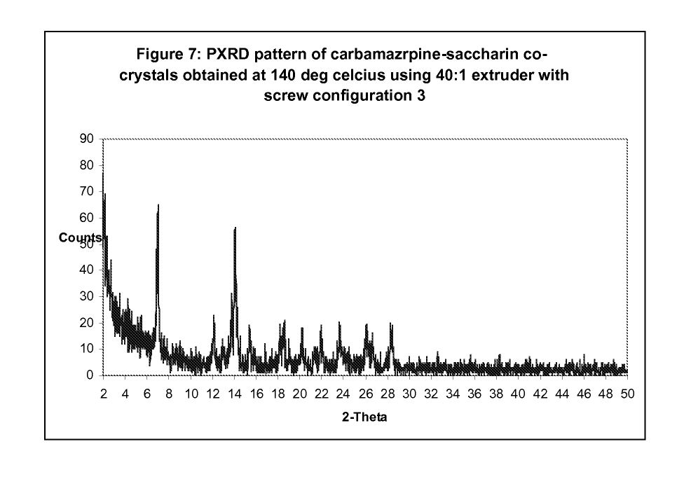

FIG. 7 shows the PXRD pattern of co-crystals of carbamazepine and saccharin obtained after extrusion using 40:1 screw configuration with feeding, distributive and dispersive zones.

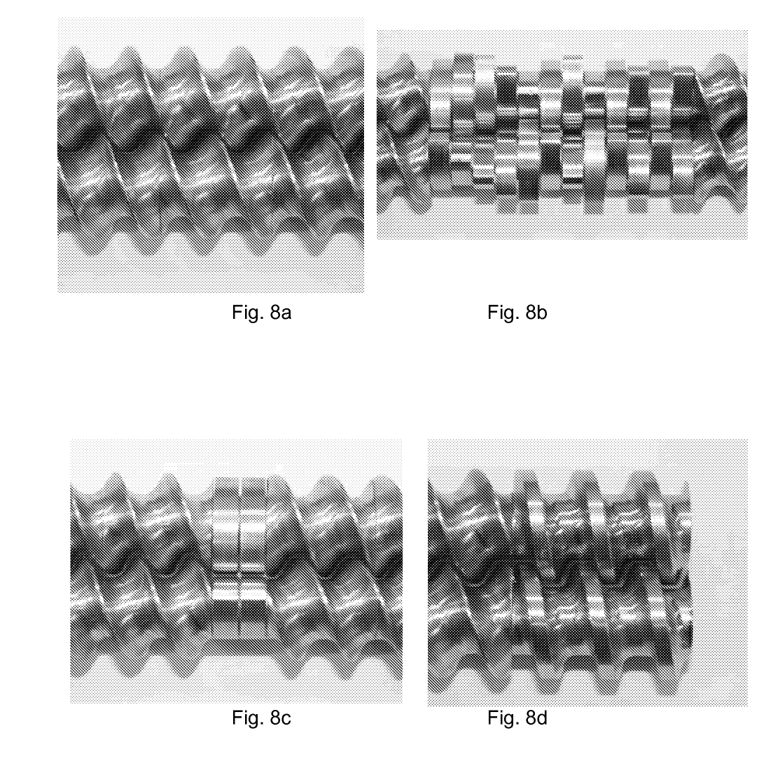

FIGS. 8a to 8d show photographs of the four types of twin screw elements as used in configurations A, B and C:

a) conveying configuration

b) staggered (i.e. distributive) mixing configuration

c) Dispersive mixing configuration

d) Discharge configuration

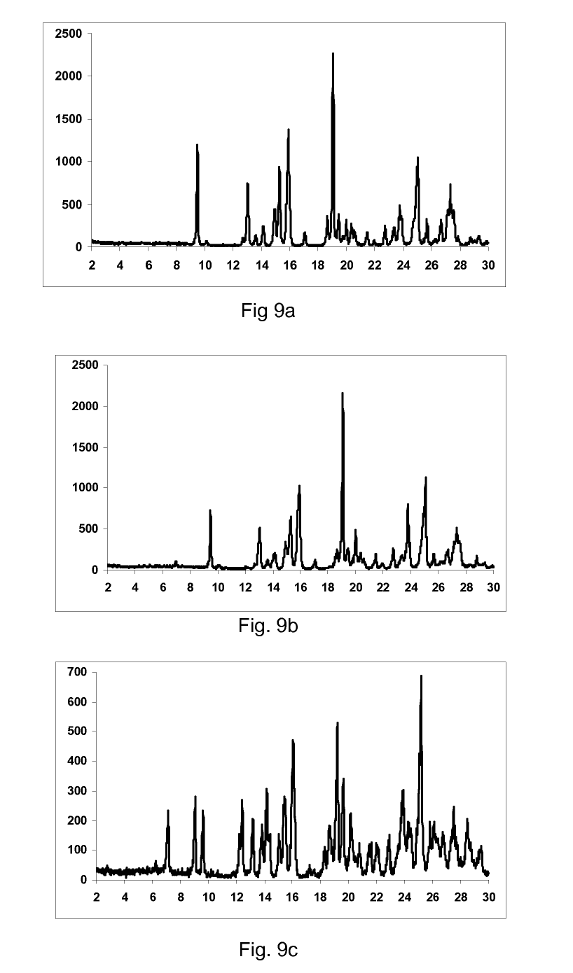

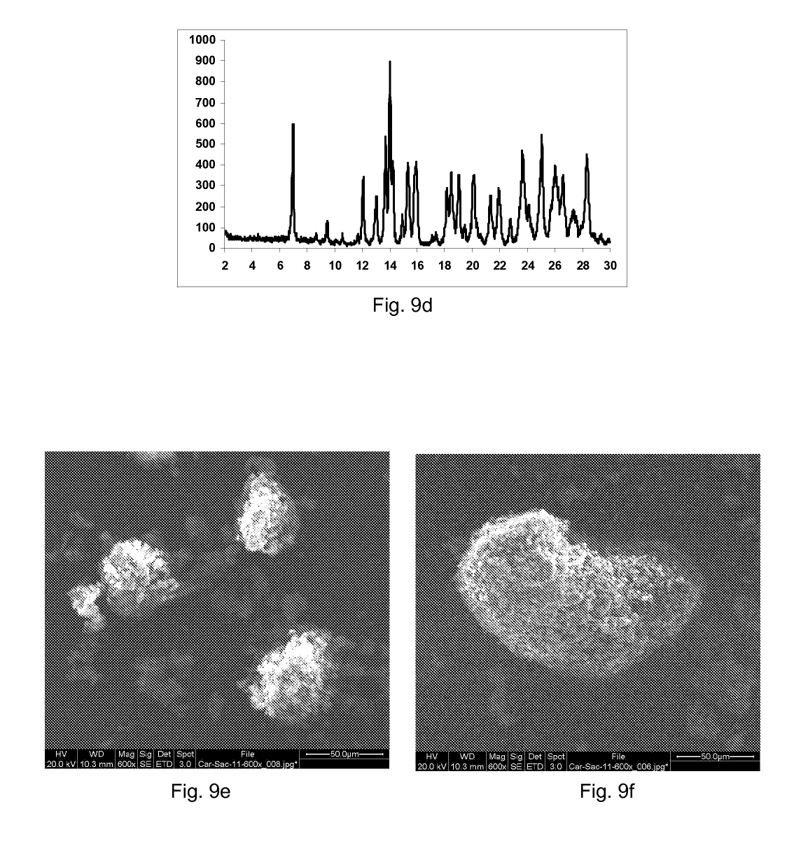

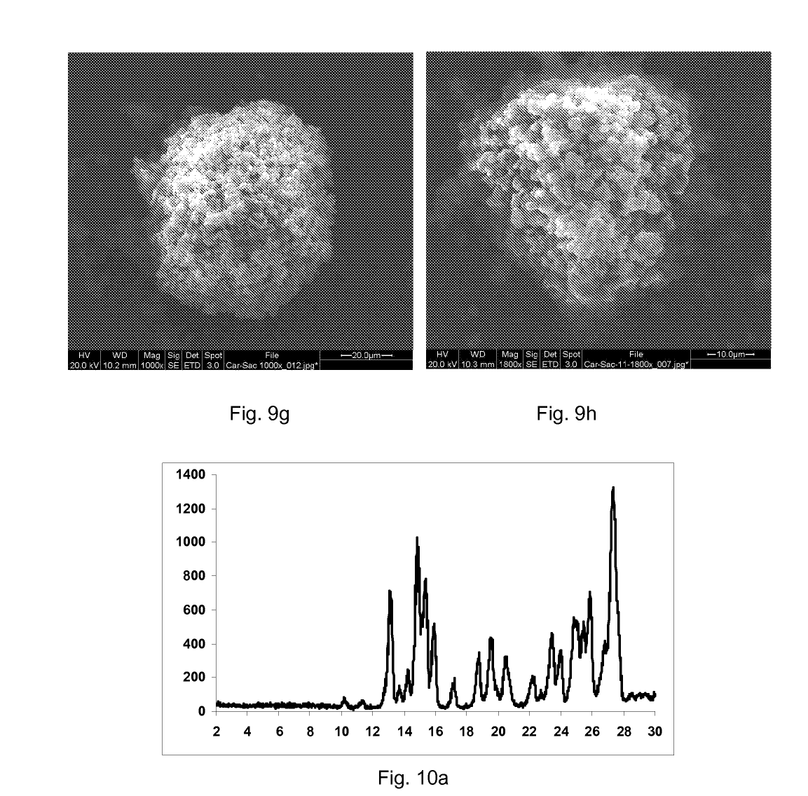

FIGS. 9a to 9d show graphs of the PXRD results following trituration or extrusion using configurations A, B or C respectively of carbamazepine and saccharin (for all the PXRD result graphs, the X axis shows 2-θ and the Y axis shows counts);

FIGS. 9e to 9h show SEM images of agglomerated carbamazepine and saccharin co-crystals produced using configuration C at 600×, 600×, 1000× and 1800× magnification respectively.

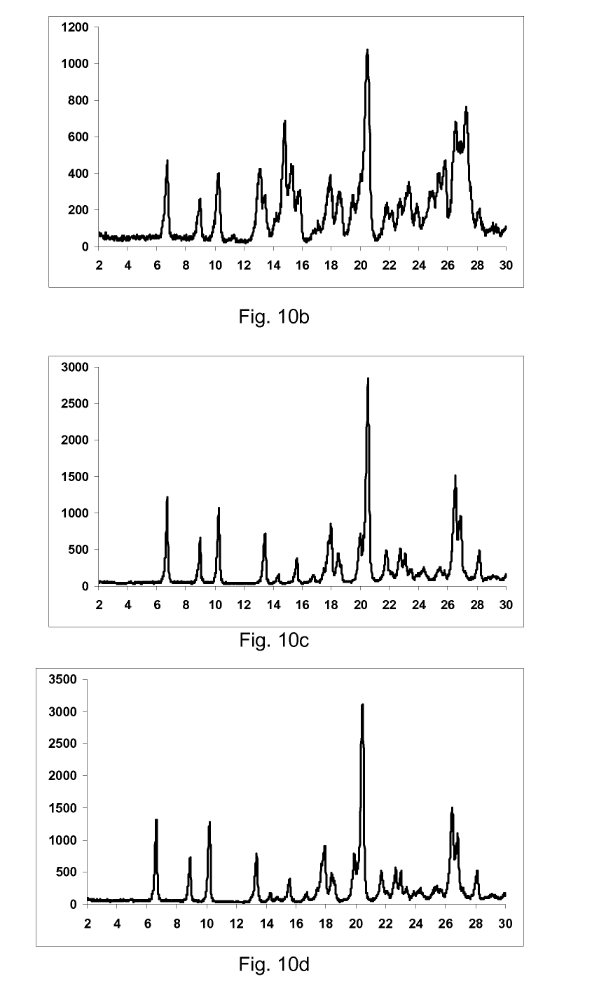

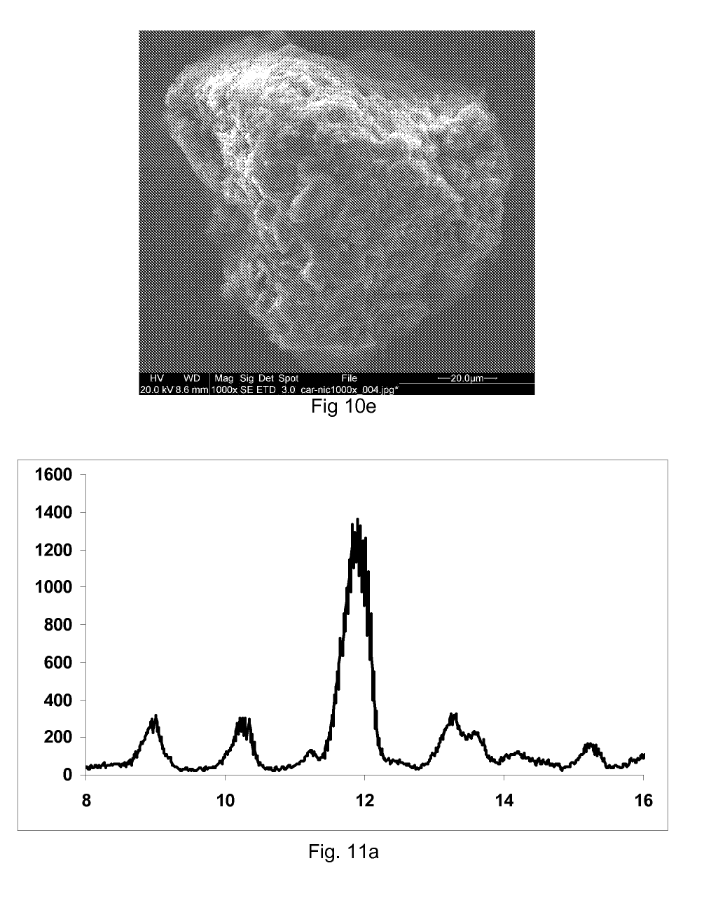

FIGS. 10a to 10d show graphs of the PXRD results following trituration or extrusion using configurations A, B or C respectively of carbamazepine and nicotinamide;

FIG. 10e shows a SEM image of agglomerated carbamazepine and nicotinamide co-crystals produced using configuration Cat 1000× magnification;

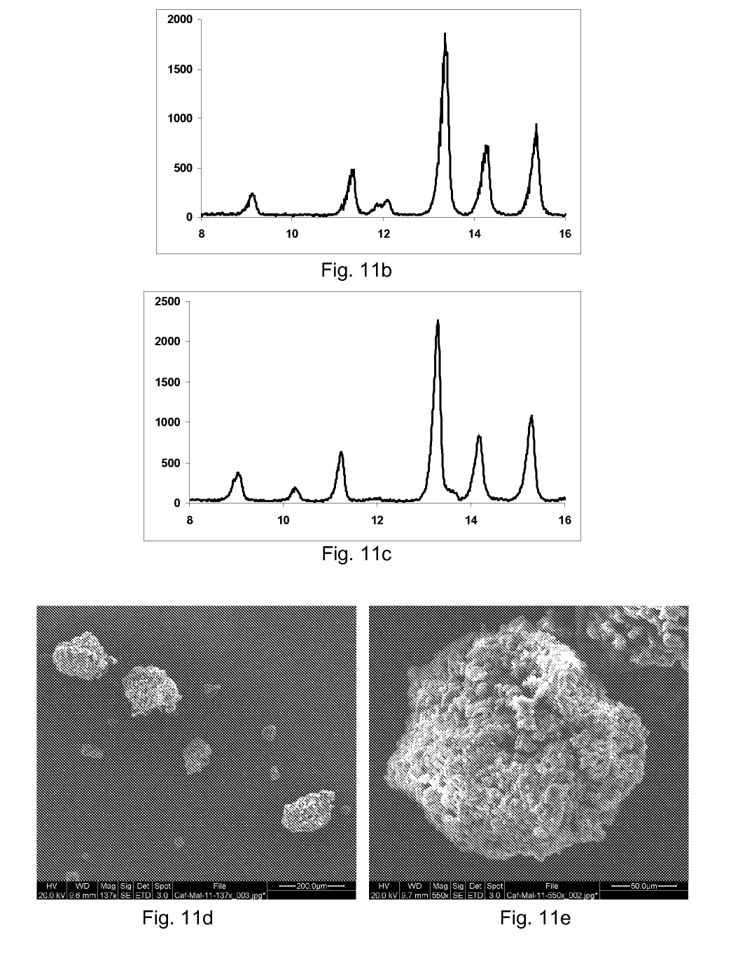

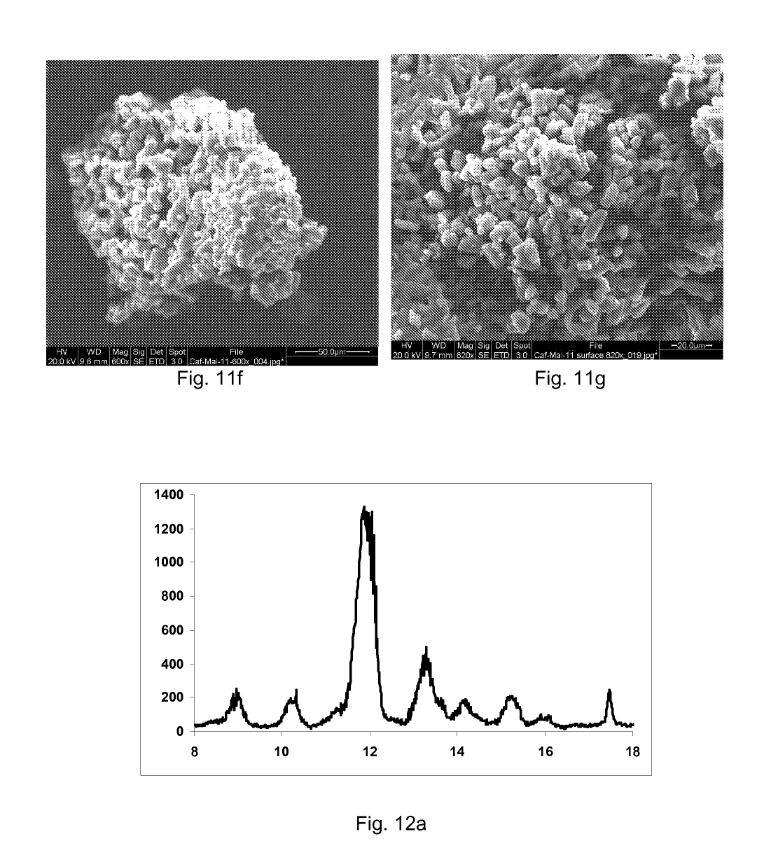

FIGS. 11a to 11c show graphs of the PXRD results following extrusion using configurations A, B or C respectively of caffeine and maleic acid (1:1 molar ratio);

FIGS. 11d to 11g show SEM images of agglomerated caffeine and maleic acid co-crystals (1:1 molar ratio) produced using configuration C at 137×, 550×, 600× and 820× magnification respectively;

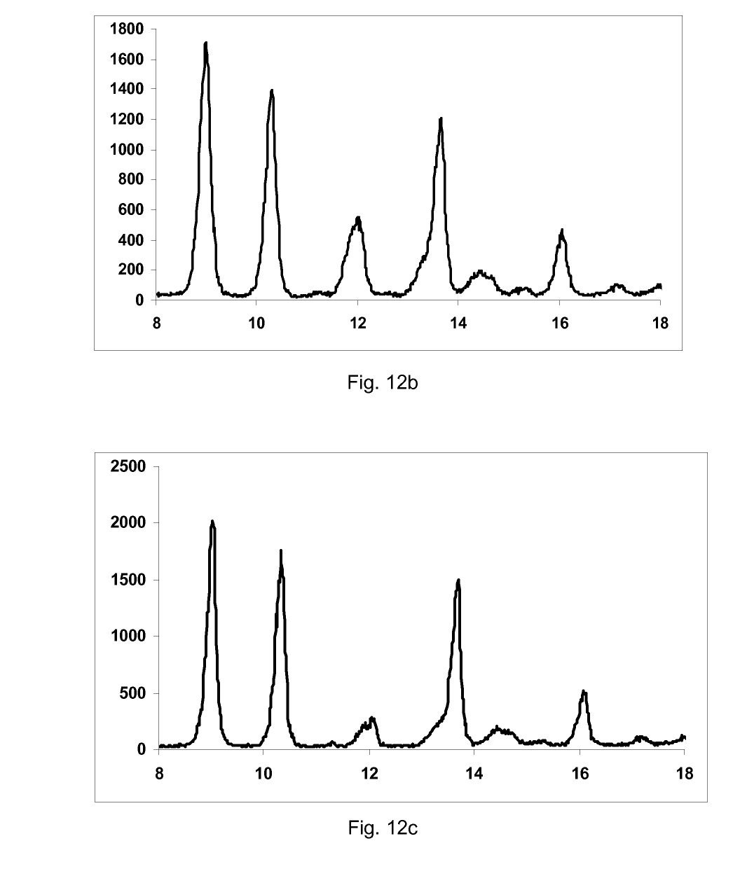

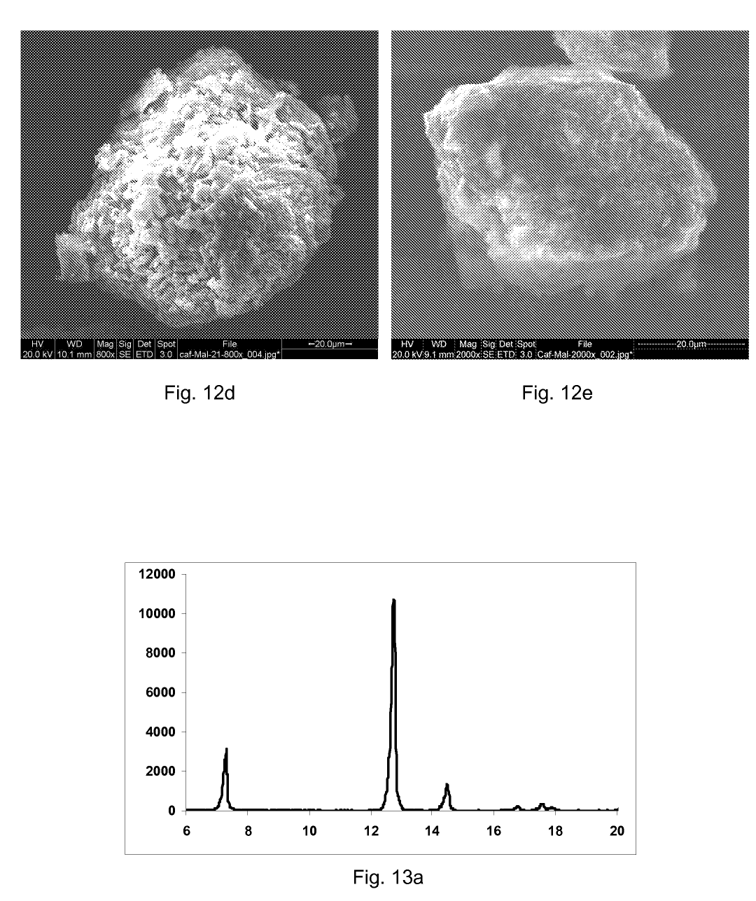

FIGS. 12a to 12c show graphs of the PXRD results following trituration or extrusion using configurations A, B or C respectively of caffeine and maleic acid (2:1 molar ratio);

FIGS. 12d and 12e show SEM images of agglomerated caffeine and maleic acid co-crystals (2:1 molar ratio) produced using configuration C at 800 and 2000× magnification respectively;

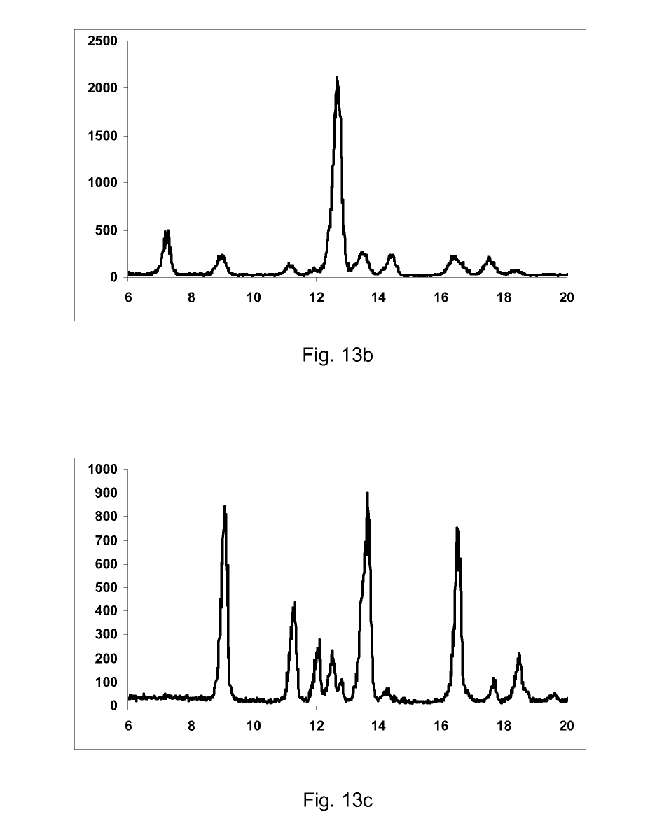

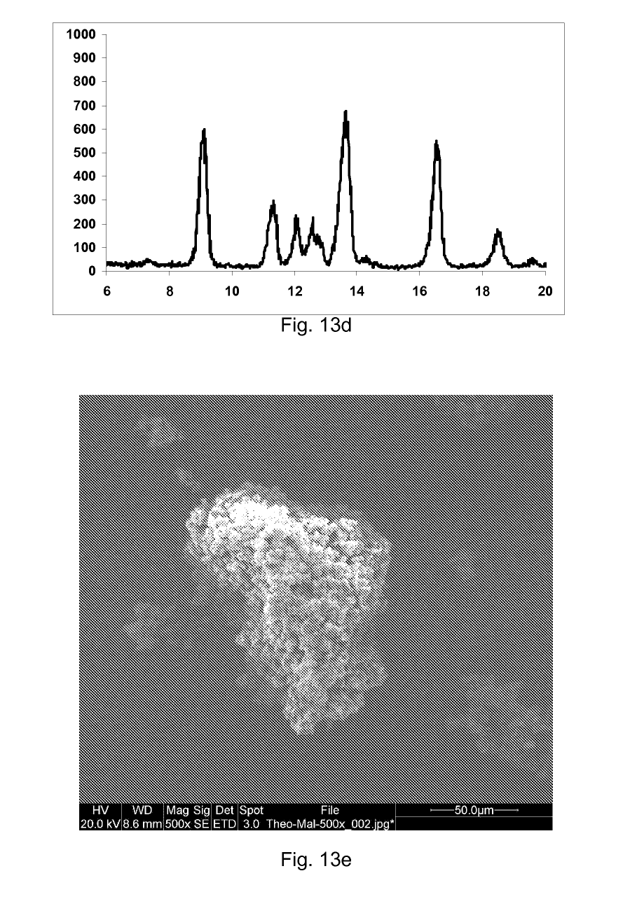

FIGS. 13a to 13d show graphs of the PXRD results following trituration or extrusion using configurations A, B or C respectively of theophylline and maleic acid;

FIG. 13e shows a SEM image of agglomerated theophylline and maleic acid co-crystals produced using configuration C at 500× magnification;

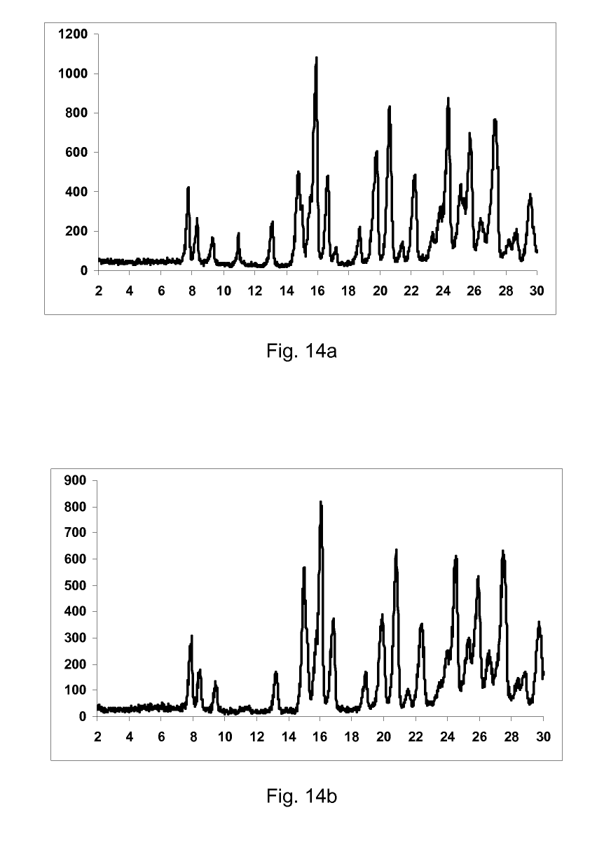

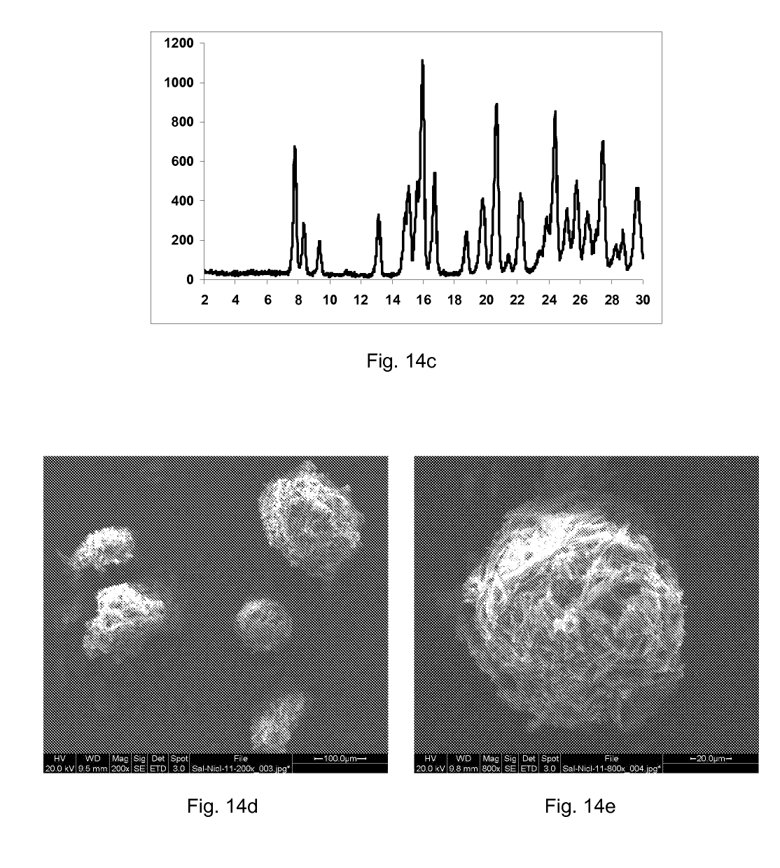

FIGS. 14a to 14c show graphs of the PXRD results following extrusion using configurations A, B or C respectively of salicylic acid and nicotinamide;

FIGS. 14d and 14e show SEM images of agglomerated salicylic acid-nicotinamide co-crystals produced using configuration C at 200× and 800× magnification respectively; and

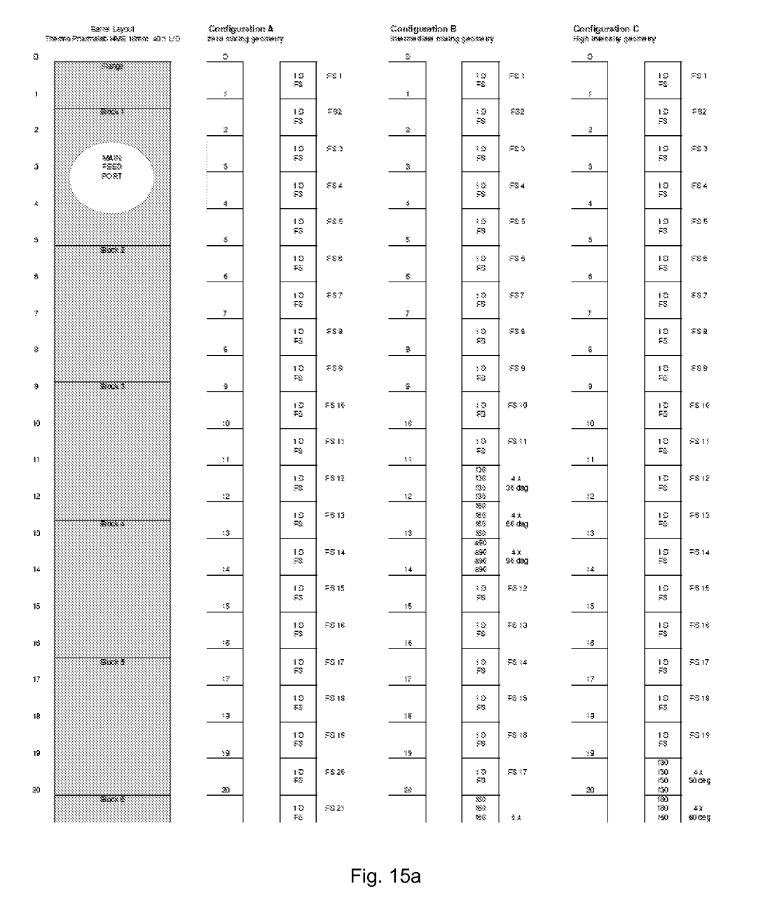

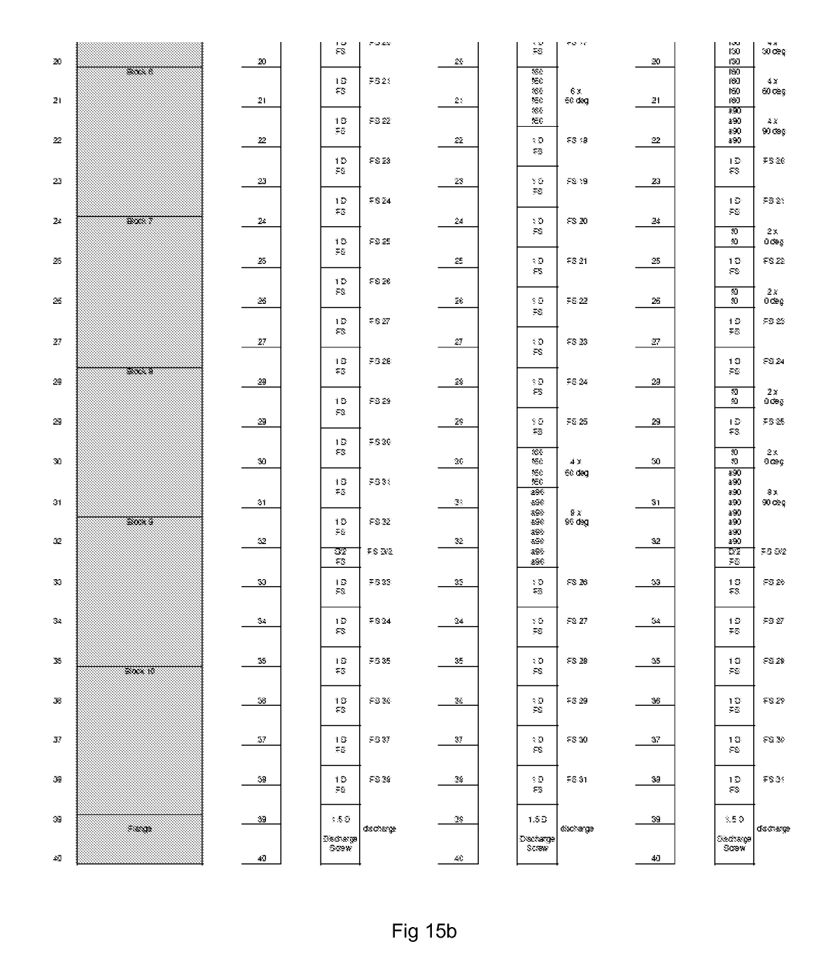

FIGS. 15a and 15b show a schematic representation of the screw configurations A, B and C against a representation of the extrusion apparatus itself.

### EXTRUSION—BACKGROUND

Extrusion can be defined as the process of forming a product by forcing material through an orifice or die. This process is normally carried out in continuous manner by the action of an Archimedean screw rotating in a heated barrel in case of melt extrusion. For polymers, melting is achieved by the dual action of conductive heating through the barrel walls and viscous shearing of the polymer. The simplest and most widely used form of extruder is that employing a single screw, which generally has a simple single flighted design to achieve melting and metering of the molten material.

Twin screw extruders (TSEs) were developed to overcome the poor mixing performance of single screw extruders by using two screws, usually arranged side by side, rotating in the same (co-rotating) or opposing (counter-rotating) directions. Screws are typically designed to be closely or fully intermeshing, i.e. the flight tips of each screw reach the root of the opposing screw, with the exception of mechanical clearance. This allows a high degree of mixing in the ‘intermesh’ region between the two screws. TSEs operate by forced conveyance rather than relying on viscous drag flow, and the self-wiping action of the screws cause the extruder to be more sanitary, with less stagnation than single screw designs. TSEs screws normally consist of hexagonal shafts on which interchangeable screw elements are arranged. This allows for a high degree of flexibility in screw design, which can be readily configured to provide a mixture of conveyance, kneading, mixing and venting, depending upon the application. TSEs are typically starve-fed and run with incompletely filled channels.

Counter-rotating extruders have lower levels of mixing but high material feed and conveying characteristics due to the material movement within the extruder. If the flights of each screw match and completely fill the channels of the other screw the material is completely prevented from rotating with the screw and thus positively moved in the axial direction. This movement is independent of material viscosity and adherence to the metal surfaces of the barrel and screw. Residence times and melt temperatures in counter-rotating TSEs are very uniform. Material between the screws is subjected to high shear forces and causes the development of high pressures, thus counter-rotating TSEs are operated at lower screw speeds than co-rotating due to the high pressures developed between the screws. Typical polymeric applications of counter-rotating TSEs include materials which are sensitive to thermal degradation and require low residence times such as PVC and wood composite polymers.

Co-rotating extruders are the most industrially significant class of TSE and tend to have closely or fully intermeshing screw designs. Screw elements are self-wiping and high screw speeds and throughputs are possible with this design. Co-rotating TSE have the ability to mix the material longitudinally as well as transversely, so material is transported from one chamber of the screw to the other, which results in excellent mixing and a high input of energy into the mixture. Co-rotating screws offer a high degree of flexibility compared to counter rotating systems. Typical configurations include a mixture of conveying, kneading and mixing elements. Barrier elements can be used to provide melt seals and regions of high and low pressure to allow injection of liquids or removal of volatiles. Typical applications of co-rotating TSE include the vast majority of plastics compounding operations, where polymeric resins are mixed with a wide range of reinforcing fillers and additives. Blending and reactive extrusion are also widely used applications. Extrudate from co-rotating TSEs is generally pelletised for use in a subsequent forming process; TSE alone is not particularly well suited to manufacture of a product due to the low head pressures generated and the inherent fluctuations in output.

Co-Crystal Formation by Twin Screw Extrusion—Experimental Methodology

Equipment

Two co-rotating twin screw extruders were used in the formation of co-crystals, both having screw diameters of 16 mm. The first was a short extruder with screw length to diameter (L:D) ratio of 15:1 (Thermo Prism TSE 16TC) incorporating 3 temperature controlled barrel zones and 1 die zone. A long extruder with L:D ratio of 40:1 (Thermo Prism Eurolab) was also used, incorporating a total of 10 temperature controlled barrel and die zones. Extruder length combined with screw design determines the residence time and the degree of mixing possible during extrusion.

One screw configuration was used in the 15:1 L:D extruder, with a simple screw design consisting of conveying elements and one distributive mixing zone. Two screw configurations were used in the 40:1 L:D extruder, one with conveying elements and 3 mainly distributive mixing sections. The second configuration employed a more complex combination of distributive and dispersive mixing zones and a reversed conveying element. This provided high residence time and harsh mixing environment. Tables 3a to 3c summarise the three screw configurations used, and photographs showing examples of the main types of screw elements are displayed in FIG. 1.

Experimental Procedure

A cleaned extruder was pre-heated to the selected processing temperature. A range of barrel temperature profiles were used, typically increasing from a cooled feed zone to a maximum mid way along the barrel and decreasing towards the die end. For the purposes of these trials the extruders were run without a die. Extruder screw rotation speed was set; a wide range of speeds can be achieved, up to 200 revolutions per minute (rpm) with the extruders used here. Typical screw rotation speeds were set at between 20 and 50 rpm. A pre-mixed blend of active agent and co-former were then introduced into the feed hopper of the extruder. Manual dosing may prove convenient for small batch sizes (typically between 10-30 g). For larger batch sizes a gravimetric feeder system can more conveniently be employed. The extruded mixture of drug and co-former was then collected at the exit of the screws, in powder, sticky mass or molten form depending upon constituents and the set operating conditions. The collected material was subsequently analysed for co-crystal formation.

During the course of experiments, the following parameters could be adjusted:
Set temperature
Screw rotation speed
Throughput
Screw design (i.e. degree of distributive and dispersive mixing)
Number of passes through the extruder

TABLE 3a
schematic representation of screw elements, Configuration 1.

| Length (diameters) | Element type |
|---|---|
| 8.5 | Forward conveying |
| 1 | 30° forward mixing |
| 1 | 60° forward mixing |
| 1 | 90° mixing |
| 2 | Forward conveying |
| 1.5 | Discharge |

TABLE 3b
schematic representation of screw elements, Configuration 2.

| Length (diameters) | Element type |
|---|---|
| 11 | Forward conveying |
| 1 | 30° forward mixing |
| 1 | 60° forward mixing |
| 1 | 90° mixing |
| 6 | Forward conveying |
| 1.5 | 60° forward mixing |
| 8 | Forward conveying |
| 1 | 60° forward mixing |
| 2 | 90° forward mixing |
| 6 | Forward conveying |
| 1.5 | Discharge |

TABLE 3c
schematic representation of screw elements, Configuration 3.

| Length (diameters) | Element type |
|---|---|
| 11 | Forward conveying |
| 1 | 30° forward mixing |
| 1 | 60° forward mixing |
| 1 | 90° mixing |
| 6 | Forward conveying |
| 1.5 | 60° forward mixing |
| 8 | Forward conveying |
| 1 | 60° forward mixing |
| 2 | 90° forward mixing |
| 6 | Forward conveying |
| 1.5 | Discharge |

### EXAMPLE 1

A physical mixture of Ibuprofen and nicotinamide was prepared by mixing 41.2 g ibuprofen and 26 g nicotinamide (molar ratio 1:1) in a turbula mixer for 30 minutes. An extruder with L:D ratio 15:1 and screw diameter 16 mm (Thermo Prism TSE 16TC) was used. This incorporated screw configuration 1, consisting primarily of forward feeding elements and a small distributive mixing zone. Detailed screw configurations have been presented earlier. Barrel temperature was set to 80° C. Once temperature was stabilized for 15 min, the physical mixture was slowly fed to the extruder and screw was rotated at 20 rpm. The finely agglomerated product was collected at the extruder exit. The residence time for the substances through the extruder was approximately 3 minutes. The powder was cooled to room temperature then subjected to powder X-ray diffractometric (PXRD) characterization.

FIG. 2 shows a PXRD pattern of physical mixture containing ibuprofen-nicotinamide. A characteristic peak of ibuprofen can be observed at 6° 2theta.

FIG. 3 shows the PXRD pattern of co-crystals obtained after extrusion using 15:1 screw configuration with feed and single distributive mixing zone. A characteristic peak of co-crystal at 3.2° 2-theta was observed which clearly indicates that co-crystals were formed during the extrusion process. The PXRD pattern also shows the appearance of a characteristic peak of ibuprofen crystals at 6° 2theta. This indicates that a portion of ibuprofen was not transferred into co-crystal form.

### EXAMPLE 2

A physical mixture of ibuprofen and nicotinamide was prepared by mixing 41.2 g ibuprofen and 26 g nicotinamide (molar ratio 1:1) in a turbula mixer for 30 minutes. An extruder with L:D ratio 40:1 and screw diameter 16 mm was used (Thermo Prism Eurolab). This incorporated screw configuration number 2, consisting of feeding and distributive mixing zones. Detailed screw configurations have been presented earlier. Barrel temperature was set to 80° C. Once temperature was stabilized for 15 min, the physical mixture was slowly fed to the extruder and screw was rotated at 20 rpm. The residence time for the substances through the extruder was approximately 20 minutes. The finely agglomerated product was collected at the extruder exit. The powder was cooled to room temperature then subjected to powder X-ray diffractometric (PXRD) characterization.

As mentioned above, FIG. 2 shows PXRD pattern of physical mixture containing ibuprofen-nicotinamide. A characteristic peak of ibuprofen can be observed at 6° 2-theta.

FIG. 4 shows PXRD pattern of co-crystals obtained after extrusion using the 40:1 screw configuration with alternative feed and distributive mixing zone, as described above. It shows appearance of characteristic peak of co-crystal at 3.2° 2-theta. This clearly indicates that co-crystals are formed during extrusion process. The PXRD pattern also shows a small characteristic peak of ibuprofen crystals at 6° 2-theta. The PXRD pattern was analyzed using suitable computational methods which indicated that the mixture contains approximately 72% co-crystals.

### EXAMPLE 3

An ibuprofen and nicotinamide physical mixture was prepared by mixing 41.2 g ibuprofen and 26 g nicotinamide (molar ratio 1:1) in a turbula mixer for 30 minutes. An extruder with L:D ratio 40:1, (Thermo Prism Eurolab) and having alternate feed, distributive and dispersive mixing screw zones with a reverse feed screw section prior to last feed zone (Configuration 3) was set at 80° C. Once temperature was stabilized for 15 min, the physical mixture was slowly fed to the extruder and screw was rotated at 20 rpm. The residence time for the substances through the extruder was approximately 33 minutes. The finely agglomerated product was collected at the end zone. The powder was cooled to room temperature then subjected to powder X-ray diffractometric (PXRD) characterization.

As mentioned above, FIG. 2 shows PXRD pattern of physical mixture containing ibuprofen-nicotinamide. A characteristic peak of ibuprofen can be observed at 6° 2theta.

FIG. 5 shows PXRD pattern of co-crystals obtained after extrusion using 40:1 screw configuration 3. It shows appearance of characteristic peak of co-crystal at 3.2° 2theta. This clearly indicates that co-crystals are formed during extrusion process. The PXRD does not show a characteristic peak of ibuprofen crystals at 6° 2theta. The PXRD pattern analysed by suitable computational methods indicated approximately 94% co-crystal content.

FIG. 6 shows the PXRD pattern obtained using the single co-crystal of ibuprofen-nicotinamide (this has been produced by solvent technique for comparison).

FIGS. 6a to 6d show scanning electron micrographs (SEM) of agglomerates of co-crystals formed from ibuprofen-nicotinamide crystals produced using screw configuration 3 (at 350×, 1100×, 1800×, and 3500× magnification).

### EXAMPLE 4

A physical mixture of carbamazepine and saccharin was prepared by mixing 47 g carbamazepine and 37 g saccharin (molar ratio 1:1) in a turbula mixer for 30 minutes. Extrusion was carried out in a TSE incorporating screw configuration number 3, consisting of feeding, distributive mixing and dispersive mixing zones. Detailed screw configurations have been presented earlier. Barrel temperature was set to 140° C. Once temperature was stabilized for 15 min, the physical mixture was slowly fed to the extruder and screw was rotated at 20 rpm. The residence time for the substances through the extruder was approximately 33 minutes. The finely agglomerated product was collected at the end zone. The powder was cooled to room temperature then subjected to powder X-ray diffractometric (PXRD) characterization.

FIG. 7 shows PXRD pattern of co-crystals obtained after extrusion using 40:1 screw configuration 3. It shows appearance of characteristic peak of carbamazepine-saccharin co-crystals at 7° 2-theta. This clearly indicates that co-crystals are formed during extrusion process.

Co-Crystal Formation by Twin Screw Extrusion—Further Experimental Methodology

Further experiments were conducted to assess the effects of shear, mixing and residence times on co-crystal yield.

All experiments for examples 5 to 10 were performed on a Thermo Pharmalab HME 16 co-rotating twin screw extruder with screw diameter 16 mm and length to diameter (L:D) ratio of 40:1.

Three screw configurations (shown schematically in FIGS. 15a and 15b) were used to assess the effect of shear, mixing and residence time on co-crystal yield. The screw configurations are hereafter referred to as A, B and C and represent low, medium and high levels of mixing respectively. The configurations are described in more detail below:

Configuration A

This provided a minimum level of mixing intensity, and consisted of purely forward conveying elements, with a metering element at the screw tips. The configuration can be tabulated as shown in the table below:

TABLE 4
Screw configuration A, ordered from feed to discharge.

| Length (diameters) | Element type |
|---|---|
| 38.5 | Forward conveying |
| 1.5 | Discharge |

Configuration B

This provided intermediate levels of distributive mixing and is typical of the type of screw configurations used in conventional polymer compounding (mixing) operations. Distributive mixing (mixing by rearranging the flow paths) was achieved here using a series of bi-lobal mixing paddles of length D/4 arranged at specified angles from the preceding element—30, 60 or 90°. These paddles are arranged in the forward conveying direction, i.e. 30° mixing paddles provide the most forward conveying, 60° provide less and 90° provide zero forward conveying action and purely mixing. The configuration can be tabulated as shown in the table below:

TABLE 5
Screw configuration B, ordered from feed to discharge.

| Length (diameters) | Element type |
|---|---|
| 11 | Forward conveying |
| 1 | 30° forward mixing |
| 1 | 60° forward mixing |
| 1 | 90° mixing |
| 6 | Forward conveying |
| 1.5 | 60° forward mixing |
| 8 | Forward conveying |
| 1 | 60° forward mixing |
| 2 | 90° forward mixing |
| 6 | Forward conveying |
| 1.5 | Discharge |

Configuration C

This provided high levels of distributive and dispersive mixing. Dispersive mixing (high shearing action to break down agglomerates) was achieved by positioning pairs of mixing paddles together at the same orientation, i.e. without a staggering angle. This effectively created a wide mixing paddle which forces more material to pass over the high shearing tips of the paddles. The configuration can be tabulated simply as shown in the table below:

TABLE 6
Screw configuration C, ordered from feed to discharge.

| Length (diameters) | Element type |
|---|---|
| 19 | Forward conveying |
| 1 | 30° forward mixing |
| 1 | 60° forward mixing |
| 1 | 90° mixing |
| 2 | Forward conveying |
| 0.5 | 0° mixing |
| 1 | Forward conveying |
| 0.5 | 0° mixing |
| 2 | Forward conveying |
| 0.5 | 0° mixing |
| 1 | Forward conveying |
| 0.5 | 0° mixing |
| 2 | 90° mixing |
| 6.5 | Forward conveying |
| 1.5 | Discharge |

FIGS. 8a to 8d show photographs of the following types of twin screw elements as used in configurations A, B and C:
a) Conveying configuration or feed screw.
b) Staggered (i.e. distributive) mixing configuration
c) Dispersive mixing configuration
d) Discharge configuration

In FIGS. 15a and 15b a schematic representation of the barrel of the extrusion apparatus is shown at the left. The length of the apparatus is broken up into 10 zones (labelled “Blocks” in the figure)—these zones correspond to the temperature zones set out in the tables below. The length is further broken up into 40 units of length, each unit corresponding to the screw diameter—hence the annotation “D”, standing for diameter. To the right, the three screw configurations A, B and C are set out schematically. Again, the diameter measurement is used to illustrate the length of each element within the configuration. FS stand for “feed screw”, i.e. the conveying screw which provided minimal mixing. Where mixing paddles are provided, the degree of rotational offset in degrees is shown numerically, as is the number of elements—each mixing paddle is 0.25 D in length. Offsets of 30, 60 or 90 degrees corresponds to distributive mixing, and an offset of 0 degrees corresponds to regions of dispersive mixing. The annotations “f” and “a” to the mixing element offset angles are not significant. All three configurations are finished with a discharge screw.

### EXAMPLE 5

### Carbamazepine:Saccharin (1:1)

Procedure:

236 g carbamazepine was mixed with 183 g saccharin (i.e. a 1:1 stoichiometric relationship) in a turbula mixer for 30 minutes. An extruder with L:D ratio 40:1 (Pharmalab HME 16, Thermo) with different screw geometries mentioned in configurations A, B or C, without a die, was set at temperatures mentioned in table 8. Once the temperature was stabilized, the blend was fed to the extruder and screw was rotated at 20 rpm. The finely agglomerated product was collected at the end of zone 10, cooled to room temperature, and subjected to analysis.

TABLE 7

| | Quantity |
|---|---|
| Ingredient | (gm) |
| Carbamazepine | 236 |
| Saccharin | 183 |

TABLE 8

| | Zone ofextruder | | | | | | | | | |
|---|---|---|---|---|---|---|---|---|---|---|
| | Die | Zone 10 | Zone 9 | Zone 8 | Zone 7 | Zone 6 | Zone 5 | Zone 4 | Zone 3 | Zone 2 |
| Temp. (° C.) | n/a | 70 | 100 | 125 | 140 | 125 | 110 | 75 | 50 | 25 |

FIGS. 9a to 9d show graphs of the PXRD results following trituration or extrusion using configurations A, B or C respectively (N.B. for all the PXRD result graphs, the X axis shows 2-Theta (θ) and the Y axis shows counts).

FIG. 9a shows substantially no co-crystals were obtained through mixture alone, as evidenced by a lack of a peak at 2-θ=7.

As shown in FIG. 9b, no co-crystals were obtained using configuration A:

Peak at 2-θ=7, characteristic of co-crystal not observed.

Peak at 2-θ=8.9 characteristic of carbamzepine dehydrate prominently observed.

As shown in FIG. 9c, low percent purity co-crystals were obtained using configuration B:

Peak at 2-θ=7, characteristic of co-crystal, but with low intensity.

Peak at 2-θ=8.9 characteristic of carbamzepine dehydrate observed.

As shown in FIG. 9d, co-crystals at high levels were obtained using configuration C:

Characteristic peak at 2-θ=7.

No peak at 2-θ=8.9 characteristic of carbamzepine dehydrate was not observed.

FIGS. 9e to 9h show SEM images of agglomerates of carbamazepine-Saccharin co-crystals (1:1) obtained using configuration C (at at 600×, 600×, 1000× and 1800× magnification respectively).

### EXAMPLE 6

### Carbamazepine:Nicotinamide (1:1)

Procedure:

236 g carbamazepine was mixed with 122 g nicotinamide (i.e. a 1:1 stoichiometric relationship) in a turbula mixer for 30 minutes. An extruder with L:D ratio 40:1 (Pharmalab HME 16, Thermo) with different screw geometries mentioned in configurations A, B or C without a die, was set at temperatures mentioned in table 10. Once the temperature was stabilized, the blend was fed to the extruder and screw was rotated at 20 rpm. The finely agglomerated product was collected at the end of zone 10, cooled to room temperature, and subjected to analysis.

TABLE 9

| | Quantity |
|---|---|
| Ingredient | (gm) |
| Carbamazepine | 236 |
| Nicotinamide | 122 |

TABLE 10

| | Zone of extruder | | | | | | | | | |
|---|---|---|---|---|---|---|---|---|---|---|
| | Die | Zone 10 | Zone 9 | Zone 8 | Zone 7 | Zone 6 | Zone 5 | Zone 4 | Zone 3 | Zone 2 |
| Temp. (° C.) | n/a | 55 | 90 | 120 | 130 | 125 | 90 | 60 | 35 | 25 |

FIGS. 10a to 10d show graphs of the PXRD results following trituration or extrusion using configurations A, B or C, respectively (for all the PXRD result graphs, the X axis shows 2-Theta (θ) and the Y axis shows counts).

As shown in FIG. 10a, substantially no co-crystals were obtained through mixture alone, as evidenced by a lack of a peak at 2-θ=20.4.

As shown in FIG. 10b, low percent purity were co-crystals obtained using configuration A.

Peak at 2-θ=20.4, characteristic of co-crystal observed, but with much lower intensity than for configuration C.

As shown in FIG. 10c, low percent purity co-crystals obtained using configuration B.

Peak at 2-θ=20.4, characteristic of co-crystal observed, but with lower intensity than for configuration C.

Peaks at 2-θ=6.6, 8.9, 10.1, 13.3, 15.5, 17.8 and 26.5 were observed;

these peaks appear to also be representative of co-crystals, but are less significant than 20.4.

As shown in FIG. 10d, good purity co-crystals were obtained using configuration C.

Peak at 2-θ=20.4, characteristic of co-crystal observed.

Peaks at 2-θ=6.6, 8.9, 10.1, 13.3, 15.5, 17.8 and 26.5 were also observed.

FIG. 10e shows a SEM of an agglomerate of carbamazepine and nicotinamide co-crystals (1:1) obtained using configuration C (1000× magnification).

### EXAMPLE 7

### Caffeine:Maleic Acid (1:1)

Procedure:

194 g caffeine was mixed with 116 g maleic acid (i.e. a 1:1 stoichiometric relationship) in a turbula mixer for 30 minutes. An extruder with L:D ratio 40:1 (Pharmalab HME 16, Thermo) with different screw geometries mentioned in configurations A, B or C without a die, was set at temperatures mentioned in table 12. Once the temperature was stabilized, the blend was fed to the extruder and screw was rotated at 20 rpm. The finely agglomerated product was collected at the end of zone 10, cooled to room temperature, and subjected to analysis.

TABLE 11

| | Quantity |
|---|---|
| Ingredient | (gm) |
| Caffeine | 194 |
| Maleic acid | 116 |

TABLE 12

| | Zone of extruder | | | | | | | | | |
|---|---|---|---|---|---|---|---|---|---|---|
| | Die | Zone 10 | Zone 9 | Zone 8 | Zone 7 | Zone 6 | Zone 5 | Zone 4 | Zone 3 | Zone 2 |
| Temp. (° C.) | n/a | 50 | 65 | 100 | 90 | 75 | 55 | 40 | 35 | 25 |

FIGS. 11a to 11c show graphs of the PXRD results following extrusion using configurations A, B or C, respectively (for all the PXRD result graphs, the X axis shows 2-Theta (θ) and the Y axis shows counts).

As shown in FIG. 11a, very low percent purity co-crystals were obtained using configuration A as PXRD patterns showed very low intensity characteristic peaks at 2-θ=9, 11.1, 13.2, 14.2 and 15.5 compared to batch at high shear, while the peak at 2-θ=12, characteristic of anhydrous β-caffeine was very high.

As shown in FIG. 11b, low percent purity co-crystals were obtained using configuration B as PXRD patterns showed characteristic peaks at 2-θ=9, 11.1, 13.2, 14.2 and 15.5, but with low intensity compared to batch at high shear, while the small peak at 2-θ=12, characteristic of anhydrous β-caffeine was observed.

As shown in FIG. 11c, good purity co-crystals were obtained using configuration C as PXRD patterns showed characteristic peaks at 2-θ=9, 11.1, 13.2, 14.2 and 15.5 with peak at 2-θ=13.2, having highest intensity.

FIGS. 11d to 11g show SEM images of agglomerates of caffeine and maleic acid co-crystals produced using configuration Cat 137×, 550×, 600× and 820× magnification respectively.

### EXAMPLE 8

### Caffeine:Maleic Acid (2:1)

Procedure:

388 g caffeine was mixed with 116 g maleic acid (i.e. a 2:1 stoichiometric relationship) in a turbula mixer for 30 minutes. An extruder with L:D ratio 40:1 (Pharmalab HME 16, Thermo) with different screw geometries mentioned in configurations A, B or C without a die, was set at temperatures mentioned in table 14. Once the temperature was stabilized, the blend was fed to the extruder and screw was rotated at 20 rpm. The finely agglomerated product was collected at the end of zone 10 and subjected to analysis.

TABLE 13

| | Quantity |
|---|---|
| Ingredient | (gm) |
| Caffeine | 388 |
| Maleic acid | 116 |

TABLE 14

| | Zone of extruder | | | | | | | | | |
|---|---|---|---|---|---|---|---|---|---|---|
| | Die | Zone 10 | Zone 9 | Zone 8 | Zone 7 | Zone 6 | Zone 5 | Zone 4 | Zone 3 | Zone 2 |
| Temperature | n/a | 70 | 90 | 110 | 90 | 80 | 65 | 55 | 35 | 25 |

FIGS. 12a to 12c show graphs of the PXRD results following extrusion using configurations A, B or C, respectively (for all the PXRD result graphs, the X axis shows 2-Theta (θ) and the Y axis shows counts).

As shown in FIG. 12a, very low percent purity co-crystals were obtained using configuration A.

Peaks at 2-θ=8.8, 10.1, 13.5 and 16, with very low intensity compared to batch at high shear.

Intensity of peak at 2-θ=12, characteristic of anhydrous β-caffeine was very high.

As shown in FIG. 12b, low percent purity co-crystals were obtained using configuration B.

Peaks at 2-θ=8.8, 10.1, 13.5 and 16 characteristic of 2:1 co-crystal observed.

Intensity of peak at 2-θ=12 characteristic of anhydrous β-caffeine was higher.

As shown in FIG. 12c, good purity co-crystals were obtained using configuration C.

Peaks at 2-θ=8.8, 10.1, 13.5 and 16 characteristic of 2:1 co-crystal observed.

Peak at 2-θ=12 characteristic of anhydrous β-caffeine was insignificant

FIGS. 12d and 12e show SEM images of agglomerates of caffeine and maleic acid co-crystals (2:1 molar ratio) obtained using configuration C (800× and 2000× magnification).

### EXAMPLE 9

### Theophylline:Maleic Acid (1:1)

Procedure:

180 g theophylline was mixed with 116 g maleic acid (i.e. a 1:1 stoichiometric relationship) in a turbula mixer for 30 minutes. An extruder with L:D ratio 40:1 (Pharmalab HME 16, Thermo) with different screw geometries mentioned in configurations A, B or C without a die, was set at temperatures mentioned in table 16. Once the temperature was stabilized, the blend was fed to the extruder and screw was rotated at 20 rpm. The finely agglomerated product was collected at the end of zone 10 and subjected to analysis.

TABLE 15

| | Quantity |
|---|---|
| Ingredient | (gm) |
| Theophylline | 180 |
| Maleic acid | 116 |

TABLE 16

| | Zone of extruder | | | | | | | | | |
|---|---|---|---|---|---|---|---|---|---|---|
| | Die | Zone 10 | Zone 9 | Zone 8 | Zone 7 | Zone 6 | Zone 5 | Zone 4 | Zone 3 | Zone 2 |
| Temp. (° C.) | n/a | 50 | 80 | 135 | 130 | 125 | 85 | 60 | 50 | 40 |

FIGS. 13a to 13d show graphs of the PXRD results following trituration or extrusion using configurations A, B or C, respectively (for all the PXRD result graphs, the X axis shows 2-Theta (θ) and the Y axis shows counts).

As shown in FIG. 13a, substantially no co-crystals were obtained through mixture alone, as evidenced by a lack of a peak characteristic of co-crystals. Peak at 2θ=12.5 is characteristic of theophylline.

As shown in FIG. 13b, very low percent purity co-crystals were obtained using configuration A.

Peaks at 2θ=9, 11.5, 13.6 and 16.8 characteristic of co-crystal observed, but with very low and insignificant peak intensity.

Peak at 2θ=12.5 characteristic of theophylline observed prominently.

As shown in FIG. 13c, low percent purity co-crystals were obtained using configuration B.

Peaks at 2θ=9, 11.5, 12, 13.6 and 16.8 characteristic of co-crystal observed, but with low intensity compared to batch at high shear.

Peak at 2θ=12.5 characteristic of theophylline insignificant.

As shown in FIG. 13d, good purity co-crystals were obtained using configuration C.

Peaks at 2θ=9, 11.5, 12, 13.6 and 16.8, characteristic of co-crystal observed.

Peak at 2θ=12.5 characteristic of theophylline insignificant.

FIG. 13e shows a SEM image of an agglomerate of theophylline and maleic acid co-crystals (1:1 molar ratio) obtained using configuration C (550× magnification).

### EXAMPLE 10

### Salicylic Acid: Nicotinamide (1:1)

Procedure:

138 g Salicylic acid was mixed with 122 g maleic acid (i.e. a 1:1 stoichiometric relationship) in a turbula mixer for 30 minutes. An extruder with L:D ratio 40:1 (Pharmalab HME 16, Thermo) with different screw geometries mentioned in configurations A, B or C without a die, was set at temperatures mentioned in table 18. Once the temperature was stabilized, the blend was fed to the extruder and screw was rotated at 20 rpm. The finely agglomerated product was collected at the end of zone 10 and subjected to analysis.

TABLE 17

| | Quantity |
|---|---|
| Ingredient | (gm) |
| Salicylic acid | 138 |
| Nicotinamide | 122 |

TABLE 18

| | Zone of extruder | | | | | | | | | |
|---|---|---|---|---|---|---|---|---|---|---|
| | Die | Zone 10 | Zone 9 | Zone 8 | Zone 7 | Zone 6 | Zone 5 | Zone 4 | Zone 3 | Zone 2 |
| Temp. (° C.) | n/a | 60 | 80 | 105 | 120 | 110 | 80 | 60 | 40 | 25 |

FIGS. 14a to 14c show graphs of the PXRD results following extrusion using configurations A, B or C, respectively (for all the PXRD result graphs, the X axis shows 2-Theta (θ) and the Y axis shows counts).

As shown in FIG. 14a, low purity co-crystals were obtained using configuration A.

Peaks at 2-θ=7.8, 8.4 and 9.1, characteristic of co-crystal observed, but with lower intensity compared to high shear extrusion.

As shown in FIG. 14b, good purity co-crystals were obtained at medium shear using configuration B.

Peaks at 2-θ=7.8, 8.4 and 9.1, characteristic of co-crystal observed.

As shown in FIG. 14c, good purity co-crystals obtained using configuration C.

Peaks at 2θ=7.8, 8.4 and 9.1, characteristic of co-crystal observed.

FIGS. 14d and 14e show agglomerates of salicylic acid and nicotinamide co-crystals formed using configuration C (200× and 800× magnification respectively).

From Examples 5 to 10 it can be seen that configuration C consistently provided co-crystals of relatively high purity. Accordingly, it can be concluded that:
Extrusion provides a reliable method for producing co-crystals from a wide variety of APIs and guest substance.
Severity of mixing is significant in achieving co-crystallisation.
Provision of regions of dispersive mixing is significant in achieving high levels of co-crystal formation.

Dissolution Data (In Vitro Drug Release):

The following experiment was performed to compare saturation solubility and in vitro dissolution of co-crystals formed according to Example 5 with those of pure carbamazepine in water and simulated gastric fluid.

Carbamazepine: Saccharin

Saturation solubility measurements of pure carbamazepine and carbamazepine-saccharin co-crystals were carried out by adding known excess amount of samples in 20 ml water. Samples were stirred at 20 rpm in a water bath (37±0.5° C.) for 24 h. Samples were then filtered through 0.45 μm membrane filter, diluted with water and then analyzed spectrophotometrically (Jasco V-630, Jasco Corporation, Japan) at 320 nm. Carbamazepine-Saccharin co-crystal (0.78 mg/ml) showed significant improvement in the saturation solubility compared to carbamazepine (0.29 mg/ml).

TABLE 19

| Sr. | | Saturation |
|---|---|---|
| No | Description | Solubility (mg/ml) |
| 1 | Carbamazepine | 0.29 |
| 2 | Carbamazepine-Saccharin | 0.78 |
| | co-crystals | |

Carbamazepine:Saccharin—In Vitro Dissolution Test

Dissolution studies on pure carbamazepine and agglomerates of carbamazepine-saccharin co-crystals produced according to Example 5 above, using configuration C, (equivalent to 200 mg of carbamazepine) were performed in 900 ml of simulated gastric fluid (SGF) using USP 24 type II dissolution test apparatus. SGF was prepared with 2 g/L NaCl and 1 g/L Triton X-100, and acidified to pH 2 with HCl. Bath temperature was maintained at 37±0.5° C. and stirred at 100 rpm. Samples were collected at 15, 30 and 60 min and replaced with a fresh dissolution medium. After centrifugation, filtration through 0.45 μm membrane filter and suitable dilution, concentration of carbamazepine was determined spectrophotometrically at 320 nm.

TABLE 20

| | Percentage of | Percentage of Carbamazepine |
|---|---|---|
| Time | Carbamazepine | released from agglomerates of |
| (Minutes) | released from pure drug | co-crystals |
| 15 | 43.48 ± 2.23 | 74.74 ± 3.23 |
| 30 | 59.29 ± 2.21 | 82.89 ± 3.21 |
| 60 | 69.11 ± 2.24 | 86.79 ± 3.26 |

Conclusion

Pure carbamazepine powder showed only 69.11% drug release during 60 minutes in SGF. Whereas, the carbamazepine-saccharin co-crystals showed 86.79% drug release. The initial drug release from the co-crystal agglomerates was faster as compared to pure drug. Co-crystals showed 74.74% drug release in 15 minutes and 82.89% drug release in 30 minutes, whilst pure carbamazepine only showed 43.48% drug release in 15 minutes and 59.29% drug release in 30 minutes. The results therefore demonstrate that the products of the present method are extremely well suited to increasing the bioavailability of active agents included in the co-crystal structure.

Compressibility Studies (Tablet Forming)

The output products of batches extruded using configuration C were analysed for their compaction and tablet forming properties.

Carbamazepine:Saccharin Co-Crystal (1:1)

The compressibility studies were carried out using compaction studies press (Caleva Process Solutions Ltd., England) fitted with 10 mm diameter flat faced punch. The die wall was cleaned with acetone and pre-lubricated with magnesium stearate before each compression. 300 mg of agglomerates of carbamazepine-saccharin co-crystals were hand filled into the die. Compression and decompression was operated at 100 mm/min, dwell load level 10,000 N, dwell time 0.1 second and the volume changes against compression force were recorded. The thickness of tablets was measured using thickness gauge (Mitutoyo, Japan) and the hardness was tested using hardness tester (Schleuniger-4M, Copley).

TABLE 21

| | Granule | | |
|---|---|---|---|
| | weight | Thickness | Hardness |
| Description | (mg) | (mm) | (Kp) |
| Agglomerates of | 300 | 2.0 ± 0.02 | 10 ± 0.02 |
| Carbamazepine-Saccharin | | | |
| co-crystals | | | |

| | | |
|---|---|---|
| Physical mixture | 300 | Tablets could not be |
| Carbamazepine-Saccharin | | compressed (heavy |
| | | lamination) |

Ibuprofen-Nicotinamide Co-Crystals (1:1):

The compressibility studies were carried out using compaction studies press (Caleva Process Solutions Ltd., England) fitted with 10 mm diameter flat faced punch. The die wall was cleaned with acetone and pre-lubricated with magnesium stearate before each compression. 400 mg of agglomerates of Ibuprofen-nicotinamide co-crystals were hand filled into the die. Compression and decompression was operated at 100 mm/min, dwell load level 5,000 N, dwell time 0.1 second and the volume changes against compression force were recorded. The thickness of tablets was measured using thickness gauge (Mitutoyo, Japan) and the hardness was tested using hardness tester (Schleuniger-4M, Copley).

TABLE 22

| | Granule | Thickness | Hardness |
|---|---|---|---|
| Description | weight (mg) | (mm) | (Kp) |
| Agglomerates of | 400 | 3.15 | 9 ± 0.02 |
| Ibuprofen-Nicotinamide | | | |
| co-crystals | | | |
| Physical mixture | 400 | 3.6 | 6 ± 0.02 |

| | |
|---|---|
| Ibuprofen-Nicotinamide | But tablets showed heavy lamination |

Conclusion

The co-crystal agglomerates produced by this technology were directly compressible, whilst the physical mixture of the components failed to form a compact under the same conditions. The compactability of the powder material which is obtained by the present invention in terms of breaking strength of the compact has shown that the compacts obtained using agglomerates of co-crystal have higher breaking strength as compared to one obtained using physical mixture.

The results of these experiments demonstrate that the product of extrusion processes as set out above are well suited for direct formation into tablets by compaction. This is extremely useful for simplifying tablet production.

1. A method of producing a co-crystal, the method comprising the steps of:
providing a first substance and a second substance, wherein the first and second substances are compatible to form a co-crystal;
mixing said first and second substances together; and
exposing the mixture of said first and second substances to prolonged and sustained conditions of pressure and shear sufficient to form a co-crystal of said first and second substance.

2. The method of claim 1 comprising the step of identifying the presence of a co-crystal by comparing the PXRD patterns of the output product of the method as set out above with the PXRD patterns of the first and second substances alone or in a mixture, or with known PXRD patterns of the co-crystal of interest.

3. The method of claim 1, wherein the method is a continuous flow method.

4. The method of claim 1, wherein the first substance is an active pharmaceutical ingredient (API).

5. The method of claim 1, wherein the first and second substances are exposed to sustained conditions of pressure and shear for at least 1 minute, preferably 2 minutes or longer, particularly 2 to 40 minutes, especially from 2 to 30 minutes.

6. The method of claim 1, wherein the method is suitable to obtain an output product which comprises at least 20% w/w co-crystal, 40% w/w co-crystal, more preferably at least 60% w/w co-crystal, especially at least 80% w/w co-crystal.

7. The method of claim 1, wherein the method is suitable to obtain an output product which comprises at least 90% w/w or higher co-crystal purity can be achieved.

8. The method of claim 1, wherein the pressure and shear are applied in an extrusion method.

9. The method of claim 1, wherein the pressure and shear are applied in a screw-based extrusion method.

10. The method of claim 9, wherein the screw-based method is a multiple screw-based extrusion method.

11. The method of claim 10, wherein the screw-based extrusion method is a twin-screw extrusion method.

12. The method of claim 11, wherein the twin-screw extrusion method is a co-rotating method.

13. The method of claim 10, wherein the screws are at least substantially intermeshing.

14. The method of claim 1, wherein the method is performed solely with the first and second substances which are capable of forming a co-crystal.

15. The method of claim 1, wherein the mixture of said first and second substances is exposed to additional heat.

16. The method of the process of claim 1, wherein the process is carried out, for at least a portion of the duration of the process, at a temperature around the melting point of the co-crystal forming substance with the lowest melting point.

17. The method of claim 1, comprising selecting one or more of the following properties of the extruder:
length of screw or barrel, ratio of length:
diameter of the screw (L/D ratio), composition of the screw elements (e.g. dispersive or distributive mixing elements, forward or reverse feed elements), depth of screw root (i.e. thread depth), screw rotation speed, feeding method (e.g. starvation feed versus flood feeding), and number of passes through the extruder.

18. The method of claim 1, wherein the ration of screw length to screw diameter (L/D ratio) is 15/1 or greater, preferably 20/1 or greater, preferably 30/1 or greater, and optionally around 40/1.

19. The method of claim 1, wherein the mixture is exposed to at least one period of distributive or dispersive mixing.

20. The method of claim 19, wherein the mixture is exposed to at least one period of dispersive mixing.

21. The method of claim 20, wherein the extrusion apparatus used in the method comprises dispersive mixing regions for at least 1/40 of the total length of the screw, preferably at least 1/30, more preferably at least 1/20 of the total length of the screw.

22. The method of claim 20, wherein there is at least one region of dispersive mixing, and the total length of all the regions of dispersive mixing is at least 1.5 screw diameters or more, preferably 2 screw diameters or more.

23. The method of claim 1, wherein each of the first and second substances is one of the following: carboxylic acids, amines, amides, sulphonamides, hydroxyl alcohols, ketones, amino acids, sugars, and heterocyclic bases.

24. The method of claim 1, wherein the first substance is one of naproxen, ibuprofen, ketoprofen, tolmetin, fenoprofen, indomethacin, salicylic acid, nabumetone, piroxicam, pioglitazone, glipizide, glimepihde, tolbutamide, warfarin, atorvastatin (statins), prazosin, captopril, nifedipine, felodipine, amlodepine, lidocaine, lamothgnine, amphetamine, metformine, fluoxetine, primaquine, caffeine, theophylline, carbamazepine, celecoxib, valdecoxib, atenolol, and propranolol.

25. The method of claim 1, any preceding claim wherein the second substance is one of glutaric acid, citric acid, fumaric acid, malonic acid, oxalic acid, benzoic acid, malic acid, maleic acid, tartaric acid, succinic acid, adipic acid, salicylic acid, cinnamic acid, anthranilic acid, hippuhc acid, tyrosine, lysine, arginine, isoleucine, tryptophan, histidine, cysteine, saccharin, fructose, mannitol, glucose, aspartame, and nicotinamide.

26. The method of claim 1 wherein the first and second substances are provided in a stoichiometric ratio.

27. The method of claim 1 wherein the first substance is a phenylalkanoic acid.

28. The method of claim 27, wherein the first substance is ibuprofen and the second substance is nicotinamide.

29. The method of claim 1, wherein the first substance is carbamazepine and the second substance is saccharin.

30. The method of claim 1, wherein the first substance is carbamazepine and the second substance is nicotinamide.

31. The method of claim 1, wherein the first substance is caffeine and the second substance is maleic acid.

32. The method of claim 1, wherein the first substance is theophylline the second substance is maleic acid.

33. The method of claim 1, wherein the first substance is salicylic acid the second substance is nicotinamide.

34. The method of claim 1 comprising the step of introducing a modifier compound into the extrusion process after co-crystallisation has substantially completed.

35. A method of forming particles comprising particles of agglomerated co-crystals comprising a method according to claim 1.

36. A method of forming a unit dose form of an active agent comprising carrying out the method according to claim 1, and compressing said particles, optionally in a suitable mould, to form the unit dose form.

37. The method of claim 36, wherein the method comprises compressing particles of agglomerated co-crystals to form the unit dose form.

38. A composition comprising a co-crystal, the co-crystal comprising a first and a second substance, wherein the first and second substances have been exposed to a process according to claim 1.

39. A product comprising a co-crystal of one or more of:
a phenylalkanoic acid and nicotinamide, preferably ibuprofen, and nicotinamide;
carbamazepine and saccharin;
carbamazepine and nicotinamide;
caffeine and maleic acid;
theophylline and maleic acid; and
salicylic acid and nicotinamide.

40. A product according to claim 39, comprising at least 50% co-crystal w/w, more preferably at least 75% co-crystal w/w.

41. A composition comprising a co-crystal obtained or obtainable by a process according to claim 1.

42. A composition according to claim 41, comprising particles of agglomerated co-crystals, preferably having a diameter of 2 to 3000 μm

43. A pharmaceutical preparation comprising a composition comprising a co-crystal according to claim 38, optionally in combination with a pharmaceutically acceptable excipient.

44. A co-crystal according to claim 38 for use in medical treatment.

45. Use of a co-crystal according to claim 38 in the manufacture of a medicament for the treatment of a medical condition.
